(19) United States
(12) Patent Application Publication
(10) Pub. No.: US 2026/0364003 A1
(43) Pub. Date: Nov. 19, 2026

(54) EYE DROP MICROSUSPENSIONS OF MTOR INHIBITORS

(71) Applicant: OCULIS OPERATIONS SARL, Lausanne (CH)

(72) Inventors: Thorsteinn LOFTSSON, Reykjavik (IS); Frédéric PILOTAZ, Nice (FR); Jón Freyr EGILSSON, Hafnarfjördur (IS)

(21) Appl. No.: 19/118,610

(22) PCT Filed: Oct. 6, 2023
(86) PCT No.: PCT/EP2023/077759

§ 371 Date: Apr. 4, 2025

(30) Foreign Application Priority Data
Oct. 7, 2022 (EP) 22200354.3

(51) Int. Cl. A61K 9/00; A61K 9/10; A61K 31/436; A61K 47/10; A61K 47/18; A61K 47/40; A61K 47/44

(52) U.S. Cl. CPC A61K 9/0048; A61K 9/10; A61K 31/436; A61K 47/10; A61K 47/183; A61K 47/40; A61K 47/44

(57) ABSTRACT

The present invention relates to an aqueous eye drop lipid microsuspension of mTOR inhibitors, e.g. everolimus, and their topical use for treating ocular disorders. More specifically, the ophthalmic composition comprises at least one mTOR inhibitor as the active pharmaceutical ingredient, preferably everolimus, a cyclodextrin, preferably alpha-cyclodextrin, and an oil, preferably castor oil. The invention further relates to the use of the aqueous eye drop lipid microsuspension in treating non-infectious ocular conjunctivitis, in preventing rejection of corneal graft or for treating dry eye disorders and meibomian gland dysfunctions.

### TECHNICAL FIELD

This disclosure pertains to the field of ophthalmic formulation. More specifically, the present disclosure relates to an aqueous eye drop lipid microsuspension of mTOR inhibitors, e.g. everolimus, and their topical use for treating ocular disorders.

### BACKGROUND ART

Macrolides with mTOR inhibition properties possess a broad spectrum of therapeutic actions, including the ability to inhibit inflammation, proliferation, angiogenesis, fibrosis, and hyperpermeability. In ophthalmology, these mTOR inhibitor macrolides are, for example, reported to alive allergic conjunctivitis, prevent rejection after corneal transplantation and improve tear production in patients with dry eye disease (DED) (Mihir Shah, et al, Invest. Ophthalmol. Vis. Sci., 58 (1): 372-385, 2017; WO2020018498 A1).

The mTOR macrolides are known to cause a variety of systemic side effects and, thus, targeted delivery of the macrolides in the form of aqueous eye drops will enhance the therapeutic usefulness of the macrolides in ophthalmology (Jorge L. Jacot, David Sherris, Potential Therapeutic Roles for Inhibition of the PI3K/Akt/mTOR Pathway in the Pathophysiology of Diabetic Retinopathy, Journal of Ophthalmology, (2011), 589813).

However, these compounds are very lipophilic with Log P values ranging from 4.2 to 5.8, and have poor aqueous solubility at room temperature, ranging from 0.004 to 0.25 mg/L.

In addition, these compounds are large macrocyclic lactones with several asymmetric carbon atoms and double bonds and are consequently very unstable in aqueous solutions (EP2402350B1; Manisha Prajapati, et al, International Journal of Pharmaceutics 586 (2020) 119579).

Temsirolimus (42-[3-hydroxy-2-(hydroxymethyl)-2-methylpropanoate]-rapamycin) and deforolimus (42-(dimethylphosphinate)-rapamycin) are sirolimus derivatives with increased solubility in water, or about 7 mg/mL at room temperature, but these derivatives do not improve the chemical stability. One topical ophthalmic formulation of tacrolimus has been successfully commercialized thanks to sufficient chemical stability, but enhanced stability has been achieved by the non-solubilization of the drug (Talymus®−1 mg/mL). The product is a conventional aqueous macro-suspension where the tacrolimus remains as solid particles, physically dispersed in the vehicle. The enhanced chemical stability is obtained by keeping the drug in a solid state. Such a formulation does not provide a good ocular tolerance and leads to reduced patient therapeutical adherence to the treatment. Additionally, this formulation does not provide for a fast ocular release of the drug due to the nature of the solid drug particles and low aqueous drug solubility.

Cyclodextrins and surface-active compounds have been used to increase chemical stability of macrolides in aqueous solutions, but the stabilization was still not adequate for the macrolides to be formulated as aqueous eye drops (Manisha Prajapati, et al, International Journal of Pharmaceutics 586 (2020) 119579).

Sirolimus was shown to be stable in microemulsion eye drops containing about 25% water, but the topical bioavailability of the drug from the microemulsion was very low (Guido Buech, Eckart Bertelmann, Uwe Pleyer, Ingo Siebenbrodt, Hans-Hubert Borchert, Formulation of sirolimus eye drops and corneal permeation studies. Journal of Ocular Pharmacology and Therapeutics, 23 (2007) 292-303). This same study also evaluated the solubility and stability of sirolimus in aqueous media containing cyclodextrins, liposomes, hydrotrope mixtures, and poloxamer gels with less success, especially regarding chemical stability of dissolved sirolimus.

Hence, there is still a need to develop stable aqueous eye drops of mTOR inhibitors, in particular everolimus.

The present disclosure provides stable aqueous eye drop microsuspension where the lipophilic drug, e.g. everolimus, is protected within oil droplets that are coated with selected cyclodextrin to form lipid microsuspension. The cyclodextrin used is selected based on its relative affinity to the macrolide drug and the oil, where the preferred cyclodextrin has high affinity for the oil (i.e., forms stable surface-active complexes with the oil) and exceptionally low or, preferably, negligible affinity for the drug. The lipid microsuspension formed can be further stabilized with polymers and/or surfactants to form physically stable formulation.

Upon dilution in aqueous media, like in the tear fluid, the microparticles are dissolved releasing the drug molecules. The concentration of water in the aqueous eye drop lipid microsuspension is generally greater than 70% and typically greater than 85%. The concentration of oil in aqueous eye drop lipid microsuspension is generally less than 20% and typically less than 10%. The rapid dissolution of the microparticles upon media dilution, the low oil content and high-water content thus ensures high ocular tolerance upon topical administration to the eye.

### SUMMARY

The present disclosure relates to an ophthalmic composition comprising,
at least one mTOR inhibitor as the active pharmaceutical ingredient, preferably everolimus,
a cyclodextrin, preferably α-cyclodextrin, and
an oil, preferably castor oil.

The following embodiments, can be optionally implemented, separately or in combination one with the others:

Embodiment 1: An ophthalmic composition comprising,
at least one mTOR inhibitor as the active pharmaceutical ingredient, preferably everolimus,
a cyclodextrin, preferably α-cyclodextrin, and
an oil, preferably castor oil.

Embodiment 2: The ophthalmic composition according to Embodiment 1, in which said mTOR inhibitor is selected from the group consisting of compounds with a macrolide structure, e.g. selected from the group consisting of everolimus, pimecrolimus, ridaforolimus, sirolimus, tacrolimus, temsirolimus, umirolimus, zotarolimus and combinations thereof, in particular everolimus.

Embodiment 3: The ophthalmic composition according to Embodiment 1 or 2, in which the concentration of said mTOR inhibitor is from 0.01 to 0.1%, in particular 0.02 to 0.08%, more particularly 0.03 to 0.07%, even more particularly 0.04 to 0.06%, for example about 0.05%, by weight based on the weight of the composition.

Embodiment 4: The ophthalmic composition according to anyone of Embodiments 1 to 3, in which the cyclodextrin is α-cyclodextrin.

Embodiment 5: The ophthalmic composition according to anyone of the Embodiments 1 to 4, in which the concentration of cyclodextrin is from 0.5 to 15%, in particular 1 to 10%, more particularly 2 to 8%, even more particularly 3 to 5%, by weight based on the weight of the composition.

Embodiment 6: The ophthalmic composition according anyone of the Embodiments 1 to 5, in which the oil is selected from the group consisting of castor oil, soya oil, corn oil, olive oil, coconut oil, peanut oil, safflower oil, linseed oil, cotton seed oil, sesame oil, tea oil, caraway oil, rosemary oil, almond oil, safflower oil, linseed oil, rapeseed oil, glycerol monooleate, glyceryl mono- and dicaprylate, glyceryl mono- and dicaprinate, and combinations thereof, preferably castor oil.

Embodiment 7: The ophthalmic composition according to anyone of the Embodiments 1 to 6, in which the concentration of oil is from 0.5 to 7%, in particular, 1 to 6%, more particularly 1.5 to 5%, even more particularly 2 to 4% of oil, by weight based on the weight of the composition.

Embodiment 8: The ophthalmic composition according to anyone of the Embodiments 1 to 7, wherein the composition is a microsuspension and wherein the oil and the cyclodextrin form microparticles in an aqueous vehicle.

Embodiment 9: The ophthalmic composition according to Embodiment 8, in which the microparticles have a diameter D50 of less than 40 μm, particularly less than 25 μm, more particularly less than 10 μm.

Embodiment 10: The ophthalmic composition according to anyone of the Embodiments 1 to 9, which further comprises a tonicity agent, preferably selected from the group consisting of salts, like potassium or sodium chloride, sugars, like sorbitol, mannitol, dextran or mannitol, or other types of polyols such as, glycerol and combinations thereof.

Embodiment 11: The ophthalmic composition according to Embodiment 10, wherein said tonicity agent is glycerol.

Embodiment 12: The ophthalmic composition according to Embodiment 10 or 11, in which the concentration of said tonicity agent is 0.1 to 8%, and/or wherein the concentration is adjusted to obtain a final isotonicity of the composition between 250 and 350 mOsmol/kg, for example about 300 mOsmol/kg.

Embodiment 13: The ophthalmic composition according to anyone of the Embodiment 1 to 12, which further comprises one or more polymers or one or more stabilizing agents.

Embodiment 14: The ophthalmic composition according to Embodiment 13, in which the polymer is selected from the group consisting of a polyoxyethylene fatty acid ester, a polyoxyethylene alkylphenyl ether, a polyoxyethylene alkyl ether, a cellulose derivative, a carboxyvinyl polymer, a polyvinyl polymer, a polyvinyl alcohol, a polyvinylpyrrolidone, a copolymer of polyoxypropylene and polyoxyethylene, tyloxapol and combinations thereof, in particular a copolymer of polyoxypropylene and polyoxyethylene.

Embodiment 15: The ophthalmic composition according to Embodiment 13 or 14, in which a polymer is poloxamer 407.

Embodiment 16: The ophthalmic composition according to anyone of the Embodiment 13 to 15, in which the concentration of said polymer is 0.1 to 3% in particular 0.4 to 1.2%, more particularly 0.4 to 1.0%, by weight based on the weight of the composition, for example about 0.8% if poloxamer 407.

Embodiment 17: The ophthalmic composition according to embodiment 16, wherein the viscosity of the composition is below 30 cP, in particular below 20 cP, more particularly between 5 and 20 cP.

Embodiment 18: The ophthalmic composition according to any one of Embodiment 13-17, which comprises a stabilizing agent selected from the group consisting of polyethylene glycol monostearate, polyethylene glycol distearate, hydroxypropyl methylcellulose, hydroxypropylcellulose, polyvinylpyrrolidone, polyoxyethylene lauryl ether, polyoxyethylene octyldodecyl ether, polyoxyethylene stearyl ether, polyoxyethylene myristyl ether, polyoxyethylene oleyl ether, sorbitan esters, polyoxyethylene hexadecyl ether, polyoxyethylene castor oil derivatives, polyoxyethylene sorbitan fatty acid esters, polyethylene glycols, polyoxyethylene stearates, carboxymethylcellulose calcium, carboxymethylcellulose sodium, methylcellulose, hydroxyethylcellulose, hydroxypropyl methylcellulose, cellulose, polyvinyl alcohol (PVA), poloxamers; poloxamines, alkyl aryl polyether sulfonate; PEG-derivatized phospholipid, PEG-derivatized cholesterol, PEG-derivatized cholesterol derivative, PEG-derivatized vitamin A, PEG-derivatized vitamin E, random copolymers of vinyl pyrrolidone and vinyl acetate, and combinations thereof.

Embodiment 19: The ophthalmic composition according to Embodiment 18, which comprises a stabilizing agent selected from the group consisting of polyoxyethylene castor oil derivatives, preferably selected polyoxyl 35 castor oil, polyoxyl 40 hydrogenated castor oil, and polyoxyl 15 hydroxystearate, more preferably polyoxyl 15 hydroxystearate.

Embodiment 20: The ophthalmic composition according to Embodiment 19, which comprises polyoxyl 15 hydroxystearate as a stabilizing agent and the concentration of said polyoxyl 15 hydroxystearate is at a concentration between 0.5 and 2%, for example about 1%, by weight based on the weight of the composition.

Embodiment 21: The ophthalmic composition according to anyone of the Embodiment 1 to 20, which further comprises a complexing agent and/or an antioxidant.

Embodiment 22: The ophthalmic composition according to Embodiment 21, in which a complexing agent is EDTA (ethylenediaminetetraacetic acid).

Embodiment 23: The ophthalmic composition according to Embodiment 22, in which the composition comprises EDTA and at least an antioxidant, for example selected from the group consisting of BHA (butylated hydroxyanisol), BHT (butylated hydroxytoluene), vitamin E TPGS.

Embodiment 24: The ophthalmic composition according to Embodiment 21, 22 or 23, in which the concentration of EDTA is 0.01 to 0.2%, particularly 0.02 to 0.1%, more particularly 0.03 to 0.07%, by weight based on the weight of the composition.

Embodiment 25: The ophthalmic composition according to anyone of Embodiments 1 to 24, which is a microsuspension comprising:
everolimus;
α-cyclodextrin;
castor oil; and,
optionally, at least glycerol,
optionally, at least one polymer, at least one stabilizing agent, and/or at least one antioxidant or complexing agent.

Embodiment 26: The ophthalmic composition according to anyone of Embodiments 1 to 25, which is a microsuspension comprising:
everolimus,
α-cyclodextrin;
castor oil;
glycerol, and,
optionally, at least one polymer and/or one stabilizing agent, and/or at least one complexing agent,
optionally at least one antioxidant.

Embodiment 27: The ophthalmic composition according to anyone of Embodiments 1 to 26, which is a microsuspension comprising:
0.01 to 0.1% of everolimus, for example 0.05%;
3 to 5% of α-cyclodextrin, for example 4.0%;
2 to 4% of castor oil, for example 3.0%;
1 to 3% of glycerol, for example 2.0%;
0.5 to 2% of a stabilizing agent, for example polyoxyl 15 hydroxystearate, typically 1.0% of polyoxyl 15 hydroxystearate;
0.4 to 1.2% of a polymer, for example poloxamer 407, typically 0.8% of Poloxamer 407;
0 to 0.07% of a complexing agent, for example disodium edetate dehydrate (EDTA), typically 0.05% of disodium edetate dehydrate (EDTA);
0 to 0.5% of an antioxidant.
wherein the % are % by weight based on the weight of the composition.

Embodiment 28: A method of preparing an ophthalmic microsuspension comprising the steps of:
a) preparing an aqueous composition, A by dissolving α-cyclodextrin into purified water;
b) preparing an oil phase composition B comprising mTOR inhibitor, e.g. everolimus; preferably said oil is castor oil,
c) optionally sterilizing the composition B;
d) adding the oil phase composition B to the aqueous composition A to obtain a mixture C; and,
e) homogenizing the mixture C to obtain a microsuspension.

Embodiment 29: The method according to Embodiment 28, wherein at least one tonicity agent is added to the composition A at step a).

Embodiment 30: The method according to anyone of the Embodiment 28 or 29, wherein at least one complexing agent, e.g. EDTA, is added to the composition A at step a).

Embodiment 31: The method according to anyone of the Embodiments 28 to 30, wherein the composition A is heated at a temperature T1 comprised between 100 °C and 130 °C; in particular 105 °C and 125 °C.

Embodiment 32: The method according to anyone of the Embodiments 28 to 31, wherein an aqueous solution of concentrated stabilizing agent, for example polyoxyl 15 hydroxystearate, is added to the composition A.

Embodiment 33: The method according to anyone of the Embodiments 28 to 32, wherein the composition A is heated at a temperature T1 for a time t comprised between 10 and 40 minutes, particularly 15 and 35 min, more particularly 20 and 30 minutes.

Embodiment 34: The method according to anyone of the Embodiments 28 to 33, wherein the composition A is cooled to a temperature T2 comprised between 10 °C and 40 °C, more particularly 15 °C and 35 °C, even more particularly 20 °C and 30 °C.

Embodiment 35: The method according to anyone of the Embodiments 28 to 34, wherein the oil phase composition B at step b) is prepared by dissolving the mTOR inhibitor, e.g. everolimus, in an oil.

Embodiment 36: The method according to anyone of the Embodiments 28 to 35, wherein the oil phase composition B at step b) is prepared by dissolving everolimus in an oil at a temperature T3 comprised between 20 °C and 50 °C, particularly 25 °C and 45 °C, even more particularly 30 °C and 40 °C. In one embodiment, the oil phase composition B at step b) is prepared by dissolving everolimus in an oil at a temperature T3 comprised between 25 °C and 35 °C.

Embodiment 37: The method according to anyone of the Embodiments 28 to 36, wherein the oil phase composition B is sterilized by filtration.

Embodiment 38: The method according to anyone of Embodiments 28 to 37, wherein an aqueous solution of concentrated polymer, e.g. poloxamer 407, is added to the microsuspension after the homogeneization step e).

Embodiment 39 The method according to anyone of the Embodiments 28 to 38, wherein a hydrochloric acid solution and/or a sodium hydroxide solution is added to the microsuspension obtained at step e) to adjust the pH of the microsuspension.

Embodiment 40: The method according to Embodiment 39, wherein the pH of the microsuspension is comprised between 4 and 8, particularly between 4.5 and 7.5, more particularly between 5 and 7, and preferably between 5.2 and 5.4.

Embodiment 41: A Method of preparing an ophthalmic composition comprising the steps of:
a) preparing a composition A by dissolving α-cyclodextrin in purified water;
b) adding at least one tonicity agent, for example, glycerol, and at least one complexing agent, for example EDTA, to the composition A;
c) autoclaving the composition A of step b) for example at a temperature of 121 °C for a time of 20 minutes;
d) adding an aqueous solution of concentrated stabilizing agent, for example polyoxyl 15 hydroxystearate, to the composition of step c);
e) dissolving the mTOR inhibitor, e.g. everolimus, in an oil, preferably castor oil, for example at a temperature comprised between 25 °C and 35 °C to obtain a composition B;
f) adding the composition B through a filter having predetermined porosity to the composition of step e) to obtain a mixture C;
g) homogenizing the mixture C to obtain a microsuspension.
h) adding an aqueous solution of concentrated polymer, for example, poloxamer 407, to the microsuspension.
i) adjusting the pH of the microsuspension to the desired pH, for example between 5.0 and 6.0, typically at a pH 5.3±0.1, for example by adding a hydrochloric acid solution and/or a sodium hydroxide solution.
j) If required, adjusting the final volume or weight of the formulation by adding water.

Embodiment 42: The method according to anyone of the Embodiments 28 to 41, wherein the filter for the filtration step has a porosity of comprised between 1 and 0.01 μm, particularly 0.7 and 0.05 μm, more particularly 0.4 and 0.1 μm, even more particularly 0.3 and 0.15 μm.

Embodiment 43: The method according to anyone of the Embodiments 28 to 42, wherein the filter for the filtration step is a PES filter having a porosity of 0.2 μm.

Embodiment 44: An ophthalmic composition obtainable by the method according to any one of Embodiments 28 to 43.

Embodiment 45: An ophthalmic composition according to any one of Embodiments 1 to 27 and 44, for use in the treatment of an ocular condition in particular for treating either allergic or atopic conjunctivitis (i.e; vernal conjunctivitis), for preventing rejection after corneal transplantation, for treating dry eye disease (DED), meibomian gland dysfunctions, pterygium, corneal endothelial disorders or corneal dystrophies or for use as a post-surgical treatment in trabeculectomy.

Embodiment 46: An ophthalmic composition according to Embodiment 45, wherein said corneal endothelial disorder is selected from a corneal dystrophy, in particular Fuchs' endothelial corneal dystrophy, posterior polymorphous dystrophy, congenital hereditary endothelial dystrophy, corneal endotheliitis, cytomegalovirus corneal endotheliitis, idiopathic corneal endothelial disorder, post-ophthalmic surgery disorder, in particular post-ophthalmic laser surgery disorder, trauma and aging.

Embodiment 47: An ophthalmic composition according to any one of Embodiments 1 to 27 and 44, for use in the prevention of rejection of corneal transplantation, wherein the composition is topically administered to the eye in an amount of 1 drop, one to six times per day.

Embodiment 48: An ophthalmic composition according to any one of Embodiments 1 to 27 and 44, for use in the prevention of rejection of corneal transplantation, wherein the composition is topically administered to the eye in an amount of 1 drop, one to 3 times per day for 2 to at least 12 months, or more, starting from 4 weeks after the surgical operation.

Embodiment 49: An ophthalmic composition according to any one of Embodiments 1 to 27 and 44, for use in the treatment of dry eye disease or meibomian gland dysfunctions possibly including blepharitis, wherein the composition is topically administered to either the eye or the eyelid in an amount of 1 drop, one to 3 times per day for 2 to at least 12 months, or more.

Embodiment 50: An ophthalmic composition according to any one of Embodiments 1 to 27 and 44, for use in the treatment of an ocular condition of the anterior segment of the eye, wherein the composition is topically administered to either the eye or the eyelid in an amount of 1 drop, one to 3 times per day for 2 to at least 12 months, or more.

Embodiment 51: An ophthalmic composition for use according to any one of Embodiments 45 to 50, wherein the composition comprises 0.01 to 0.1%, in particular 0.02 to 0.08%, more particularly 0.03 to 0.07%, even more particularly 0.04 to 0.06% of mTOR inhibitor, e.g. everolimus, by weight based on the weight of the composition.

Embodiment 52: An ophthalmic composition according to any one of Embodiments 1 to 27 and 44, which is an aqueous eye drop lipid microsuspension for topical administration of mTOR inhibitor to the eye.

Embodiment 53: An ophthalmic composition according to any one of Embodiments 1 to 27 and 44, which is an aqueous eye drop lipid microsuspension for topical administration of mTOR inhibitor to the eye or eyelid of a subject in need thereof.

Embodiment 54: A method for reducing an ocular inflammation and/or cellular immunity in a subject in need thereof, said method comprising topically administering an efficient amount of a composition according to any one of Embodiments 1 to 27, 44, 52 and 53, in either the eye or the eyelid of the said subject.

Embodiment 55: A method of treating an ocular disorder, in particular for treating allergic conjunctivitis, for preventing rejection after corneal transplantation, for treating dry eye disease (DED) or meibomian gland dysfunctions in a subject in need thereof, said method comprising topically administering to either the eye or the eyelid of said subject, a therapeutically effective amount of a composition according to any one of claims 1 to 27 and 44, 51 and 52.

Embodiment 56: A composition comprising:
at least one mTOR inhibitor as the active pharmaceutical ingredient, preferably everolimus,
a cyclodextrin, preferably α-cyclodextrin, and
an oil, preferably castor oil,

Embodiment 57 The composition of embodiment 56 for topical administration to the skin, in particular eyelid, of a subject in need thereof.

Embodiment 58: The composition of Embodiment 56 or 57, for treating psoriasis, atopic dermatitis and/or other skin diseases sensitive to anti-inflammatory drugs and/or immunosuppressor drugs, in particular autoimmune skin disorders or lesions.

### BRIEF DESCRIPTION OF THE DRAWINGS

Other features, details and advantages will be shown in the following detailed description and on the figures, which show:

FIG. 1. Phase-solubility diagram of everolimus in aqueous cyclodextrin solutions: α-cyclodextrin (•), β-cyclodextrin (▪), γ-cyclodextrin (□), and 2-hydroxypropyl-γ-cyclodextrin (∘). The pH of the unbuffered solution was 4.3±0.1 (SD; γ-cyclodextrin).

FIG. 2. Semi-Log plot of the percent everolimus remaining in solution versus time: pH 4 (♦), pH 5 (∘), pH 6 (□), pH 7 (▪), and pH 8 (•).

FIG. 3. The effect of mixing speed with a high shear homogenizer probe (Ultra-Turrax® T25 homogenizer, IKA-Werke, Germany, with probe IKA-S-25N-18G), operated for constant time of 10 minutes, on the particle size.

FIG. 4. The effect of mixing time with a high shear homogenizer probe (Ultra-Turrax® T25 homogenizer, IKA-Werke, Germany, with probe IKA-S-25N-18G), operated at 8,000 rpm, on the particle size.

FIG. 5. Degradation of everolimus in aqueous solutions at various pH and 40 °C.

FIG. 6. Concentration of everolimus before (•) and after (∘) heating in an autoclave at 121 °C for 20 minutes.

FIG. 7. Particle size distribution profile from the sample manufactured with L5M Silverson homogenizer as determined by Mastersizer analyzer (see Example 1).

### DETAILED DESCRIPTION

### Definitions

As used herein, the term “about” is used herein to mean approximately, in the region of, roughly, or around. When the term “about” is used in conjunction with a numerical range, it modifies that range by extending the boundaries above and below the numerical values set forth. In general, the term “about” or “approximately” is used herein to modify a numerical value above and below the stated value by a variance of 10% or at least the variance associated to the measuring device to obtain the numerical value.

The term “dissolved” or “substantially dissolved” is used herein to mean the solubilization of a solid in a solution. It can be considered that a solid is “dissolved” or “substantially dissolved” in a solution when the resulting solution is clear or substantially clear.

The term “clear” is used herein to mean a translucent or a subtranslucent solution. Thus, a “clear” solution has a turbidity measured according to ISO standards of 100 Nephelometric Turbidity Units (NTUs), preferably 50 NTUs.

The term “substantially clear” is used herein to mean a translucent or a subtranslucent solution. Thus, a “substantially clear” solution has a turbidity measured according to ISO standards of 100 Nephelometric Turbidity Units (NTUs).

As used herein the term “% by weight of a compound X based on the weight of the composition”, also abbreviated as “% w/w”, corresponds to the weight of compound X introduced in the composition, expressed as a percentage of the total weight of the composition.

The term “microsuspension” is intended to mean a composition comprising solid complex microparticles suspended in a liquid phase. An aqueous lipid microsuspension refers to a composition comprising solid complex microparticles containing oil, suspended in an aqueous phase. The lipid microparticles have higher density than water and, thus, will form a sediment upon storage.

As used herein, the term “aqueous eye drop lipid microsuspension” refers to a microsuspension where the active pharmaceutical ingredient, e.g. everolimus, is contained in a lipid droplet coated with cyclodextrin, thereby forming solid complex microparticles suspended in an aqueous vehicle, which is suitable for use as an eye drop, i.e. for topical administration to the eye or to the skin, e.g. eyelid, of a subject.

As used herein the term “microparticle” refers to a particle having a diameter D50 of 1 μm or greater to about 200 μm. The term “nanoparticle” refers to a particle having a diameter D50 of less than 1 μm.

As used herein an “ocular condition” is a disease, ailment or other condition which affects or involves the eye, one of the parts or regions of the eye, or the surrounding tissues such as the lacrimal glands. Broadly speaking, the eye includes the eyeball and the tissues and fluids which constitute the eyeball, the periocular muscles (such as the oblique and rectus muscles), the portion of the optic nerve which is within or adjacent to the eyeball and surrounding tissues such as the lacrimal glands and the eye lids.

As used herein an “anterior ocular condition” is a disease, ailment or condition which affects or which involves an anterior (i.e. front of the eye) ocular region or site, such as a periocular muscle, an eye lid, lacrimal gland or an eye bail tissue or fluid which is located anterior to the posterior wall of the lens capsule or ciliary muscles.

Thus, an anterior ocular condition primarily affects or involves one or more of the following: the conjunctiva, the cornea, the anterior chamber, the iris, the lens, or the lens capsule, and blood vessels and nerves which vascularize or innervate an anterior ocular region or site. An anterior ocular condition is also considered herein as extending to the lacrimal apparatus. In particular, the lacrimal glands which secrete tears, and their excretory ducts which convey tear fluid to the surface of the eye.

Moreover, an anterior ocular condition affects or involves the posterior chamber, which is behind the retina but in front of the posterior wall of the lens capsule.

A “posterior ocular condition” is a disease, ailment or condition which primarily affects or involves a posterior ocular region or site such as the choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site.

Thus, a posterior ocular condition can include a disease, ailment or condition such as, for example, macular degeneration (such as non-exudative age-related macular degeneration and exudative age-related macular degeneration); choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitic retinal disease; retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma.

An anterior ocular condition includes a disease, ailment or condition such as, for example, aphakia; pseudophakia; astigmatism; blepharospasm; cataract; conjunctival diseases; non-infectious conjunctivitis including allergic conjunctivitis; atopic ocular conjunctivitis possibly including vernal conjunctivitis; corneal diseases; corneal ulcer; dry eye syndromes; eyelid diseases possibly including blepharitis; lacrimal apparatus diseases; lacrimal duct obstruction; myopia; presbyopia; pupil disorders; refractive disorders, strabismus and meibomian gland dysfunctions. As used herein, anterior ocular condition more specifically includes: corneal graft rejection prevention, dry eye disease, allergic conjunctivitis, meibomian gland dysfunctions, severe keratoconjunctivitis in general and vernal conjunctivitis more specifically. Ocular blepharitis is sometimes associated to meibomian gland dysfunctions. In that context, therapeutical treatments of either meibomian gland dysfunctions or blepharitis can be administered topically on the eyelid too.

The present description is concerned with and directed to ophthalmic compositions for topical drug delivery to the eye(s) and to methods for the treatment of an ocular condition, such as an anterior ocular condition or a posterior ocular condition or an ocular condition which can be characterized as both an anterior ocular condition and a posterior ocular condition, preferably an anterior ocular condition such as corneal graft rejection. In a specific embodiment, the ophthalmic composition can be administered topically on the eyelid.

### The Ophthalmic Composition of the Present Disclosure

The present disclosure relates to an ophthalmic composition comprising
at least one mTOR inhibitor as the active pharmaceutical ingredient, preferably everolimus,
a cyclodextrin, preferably α-cyclodextrin, and,
an oil, preferably castor oil.

More specifically, the inventors have successfully designed aqueous ophthalmic microsuspensions composed of water-insoluble lipid microparticles suspended in an aqueous vehicle. The lipid microparticles essentially consist of oil coated by surface-active lipid/cyclodextrin complexes.

In specific embodiments, the microparticles of the microsuspension have a diameter D50 of about 1 μm to about 40 μm, for example about 1 μm to about 25 μm, more particularly about 1 μm to about 10 μm. Such microsuspensions are suitable for use in both human and animal, e.g. mammalian subject as eye drop formulations.

Hence, in a preferred embodiment of the present disclosure, the composition is an aqueous eye drop lipid microsuspension, comprising mTOR inhibitor as, preferably everolimus, which comprises lipid microparticles of oil, preferably castor oil, suspended in an aqueous vehicle, and wherein the lipid microparticle is coated with α-cyclodextrin.

In preferred embodiments, the composition of the present disclosure, typically the aqueous eye drop lipid microsuspension is sterile.

In more specific embodiments, the composition is an aqueous eye drop lipid microsuspension which has shelf-life at least over 2 years in a refrigerator (i.e. stored at 4 to 8 °C), and at least over 4 weeks at room temperature (i.e. at 25 °C).

### Active Pharmaceutical Ingredient

The ophthalmic aqueous eye drop lipid microsuspensions are particularly useful for formulating eye drop microsuspensions of mTOR inhibitors, more specifically mTOR inhibitors with a macrolide structure.

mTOR inhibitors are a class of drugs that inhibit the mechanistic target of rapamycin (mTOR), which is a serine/threonine-specific protein kinase that belongs to the family of phosphatidylinositol-3 kinase (PI3K) related kinases (PIKKs). mTOR regulates cellular metabolism, growth, and proliferation by forming and signaling through two protein complexes, mTORC1 and mTORC2. The most established mTOR inhibitors are so-called rapalogs (rapamycin and its analogs), which have shown immunosuppressive activity and are used to prevent the rejection of organ and bone marrow transplants in the body.

In specific embodiments, those mTOR inhibitors include a macrolide ring, typically macrocyclic 14, 15 or 16-membered lactone ring.

mTOR inhibitors for use in the aqueous eye drop lipid microsuspension of the present disclosure includes without limitation, everolimus, pimecrolimus, ridaforolimus (also called deforolimus), sirolimus (also called rapamycin), tacrolimus, temsirolimus, umirolimus, and zotarolimus, and combinations thereof.

In a preferred embodiment, everolimus is used as the active pharmaceutical ingredient, either as a sole active pharmaceutical ingredient in the microsuspension or in combination with other drugs.

Everolimus is currently used both as an antineoplastic and immunosuppressant drug. In particular. Everolimus is approved as active ingredient in particular for treating kidney, pancreas and breast cancers and for preventing graft rejection, in particular for hear, kidney and liver grafts.

The formula of everolimus is shown below:

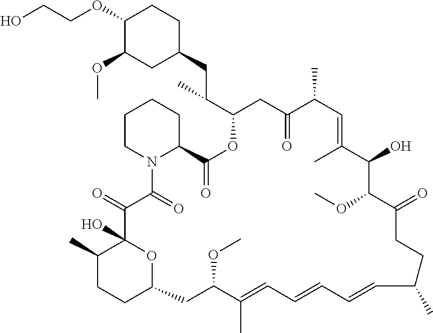 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)CC[C@H](C)/C=C/C=C/C=C/1C]

In specific embodiments, the mTOR inhibitors, preferably everolimus, are comprised in an amount between 0.01% and 0.1%, for example 0.05% in the aqueous eye drop lipid microsuspension.

In specific embodiments, the mTOR inhibitors, preferably everolimus, are stable in the aqueous eye drop lipid microsuspension of the disclosure for at least 720 days, i.e. its amount is decreased by less than 10% after 720 days at 5 °C, as measured by reversed phase liquid chromatography (HPLC) as described in Example 1.

The inventors have also shown that the eye drop lipid microsuspension of the present disclosure can be used with APIs other than mTOR inhibitors. The properties of the microsuspension are indeed comparable when the microsuspension is used with a wide variety of drugs, as illustrated in Example 20. The composition can thus be used as a vehicle for delivering a drug to the eye, such as a corticosteroid.

In one aspect, the present disclosure relates to an ophthalmic composition comprising
at least one active pharmaceutical ingredient,
a cyclodextrin, preferably α-cyclodextrin, and,
an oil, preferably castor oil.

The composition is particularly suited as a vehicle for lipophilic drugs, in particular drugs with Log P values of 1.9 or higher, in particular Log P values of 3 or higher, e.g. drugs with Log P values ranging from 4 to 8. Preferably, the active pharmaceutical ingredient is an anti-inflammatory compound. In specific embodiments, the active pharmaceutical ingredient is selected from a mTOR inhibitor, a cyclosporin and a corticosteroid. In specific embodiments, the corticosteroid is selected from loteprednol etabonate and dexamethasone. In specific embodiments, the active pharmaceutical ingredient is selected from everolimus, tacrolimus, sirolimus, cyclosporin A, loteprednol etabonate and dexamethasone.

### Cyclodextrin

The ophthalmic aqueous eye drop lipid microsuspension of the present disclosure comprises cyclodextrin. In specific embodiments, the cyclodextrins form lipid/cyclodextrin complexes thereby providing a coating of the oil microparticles of the aqueous eye drop lipid microsuspension of the present disclosure.

Cyclodextrins are cyclic oligosaccharides containing 6 (α-cyclodextrin), 7 (β-cyclodextrin), and 8 (γ-cyclodextrin) glucopyranose monomers linked via α-1,4-glycoside bonds. α-Cyclodextrin, β-cyclodextrin and γ-cyclodextrin are naturel products formed by microbial degradation of starch.

All three natural cyclodextrins (α-cyclodextrin, β-cyclodextrin and γ-cyclodextrin) and numerous cyclodextrin derivatives can form inclusion complexes with glycerides although their affinity may differ depending on type of triglycerides, diglycerides and monoglycerides, as well as other excipients present in the aqueous media.

A preferred cyclodextrin for use in the present composition, more specifically when the mTOR inhibitor is everolimus, is α-cyclodextrin or a derivative of α-cyclodextrin which has an affinity for everolimus which is identical to α-cyclodextrin or lower. It is indeed preferable that the cyclodextrin has a low affinity to the active principle, e.g. everolimus, but a good affinity with the oil, so that everolimus is maintained in the lipid microparticles. In some embodiments, the α-cyclodextrin or a derivative thereof is selected from carboxyalkyl-α-cyclodextrin, hydroxyalkyl-α-cyclodextrin, sulfoalkylether-α-cyclodextrin, alkyl-α-cyclodextrin, and combinations thereof.

The amount of cyclodextrin, preferably α-cyclodextrin in the aqueous lipid microsuspension of the disclosure may be present at about 1 to about 10%, in particular about 2 to about 8%, by weight of cyclodextrin based on the weight of the composition.

In certain embodiments with everolimus as the mTOR inhibitor, the amount of cyclodextrin, typically alpha-cyclodextrin, in the aqueous eye drop lipid microsuspension is present at about 3% to about 5% and the amount of everolimus is about 0.01% to about 0.1%. In other embodiments, the amount of cyclodextrin, typically alpha-cyclodextrin in the aqueous eye drop lipid microsuspension is about 4.0%, in particular in combination with an amount between 0.01% to 0.1% of everolimus, for example with about 0.05% of everolimus.

Commercially available pure cyclodextrins are hygroscopic and contain significant amounts of water. For example, commercially available α-cyclodextrin contains 10.2% (w/w) water. Thus, as used herein, the weight values and % (w/w) given are based on the dry weight of cyclodextrin.

### Oils

The microparticles of the microsuspension are formed of cyclodextrin complexes with oil. The oil may be selected from one or more medium-chain or long-chain triglycerides, diglycerides, and monoglycerides that are generally available in the pharmaceutical field.

For examples, the glycerides may be selected from the group consisting of castor oil, soya oil, corn oil, olive oil, coconut oil, peanut oil, safflower oil, linseed oil, cotton seed oil, sesame oil, tea oil, caraway oil, rosemary oil, almond oil, safflower oil, linseed oil, rapeseed oil, glycerol monooleate, glyceryl mono- and dicaprylate, glyceryl mono- and dicaprinate, as well as other lipophilic solvents such as silicone oils, mineral oils and semi-fluorinated alkanes such as 1-(perfluorobutyl) pentane and 1-(perfluorohexyl) octane. Preferred oils are those that can form multiple hydrogen bonds (i.e., H-bonds) with the macrolides, like oils from vegetable origin.

In an embodiment of the present disclosure, the oil is selected from the group consisting of castor oil, soya oil, corn oil, olive oil, coconut oil, peanut oil, safflower oil, linseed oil, cotton seed oil, sesame oil, tea oil, caraway oil, rosemary oil, almond oil, safflower oil, linseed oil, rapeseed oil, glycerol monooleate, glyceryl mono- and dicaprylate, glyceryl mono- and dicaprinate, and combinations thereof, preferably castor oil. Possible oils are highly refined oils with low acid value and peroxide value as determined in the European Pharmacopoeia 11.0 (sections 2.5.1 and 2.5.5). Example of such highly purified oil is Super Refined™ Castor Oil from Croda (www.crodapharma.com).

In certain embodiments, the peroxide value of the oil is from 0 to 5 meqO₂/kg, in particular from 0 to 2.5 meqO₂/kg, more particularly from 0 to 1 meqO₂/kg.

In certain embodiments, the acid value of the oil is from 0 to 1 mg KOH/g, in particular of about 0.8 mg KOH/g

In certain embodiments with everolimus as the mTOR inhibitor, the oil is castor oil.

In certain embodiments with everolimus as the mTOR inhibitor, the amount of oil, preferably castor oil, in the aqueous eye drop lipid microsuspension is from 2% to 4% w/w, and the amount of everolimus is between 0.01% and 0.1% w/w. In other embodiments, the amount of oil, typically castor oil in the aqueous eye drop lipid microsuspension may be about 3.0% w/w, in particular in combination with an amount of about 0.01% to about 0.1% of everolimus, for example about 0.05% w/w of everolimus.

In certain embodiments, the mole (or molar) ratio of the amount of cyclodextrin or derivative thereof, to the amount of the mTOR inhibitor is at least about 2:1 to about 500:1.

In certain embodiments, the mole (or molar) ration of the amount of α-cyclodextrin or derivative thereof, to the amount of everolimus as the active pharmaceutical ingredient is at least about 2:1 to about 500:1, for example between 40:1 and 120:1, more preferably between 70:1 and 90:1 and for example about 80:1.

### Tonicity Agent

In some embodiments, the ophthalmic compositions have one or more tonicity agents which can be used to adjust the tonicity of the composition, in particular the aqueous eye drop lipid microsuspension as disclosed herein, more preferably with everolimus as the active pharmaceutical ingredient.

Suitable tonicity agents include without limitations, salts, like potassium or sodium chloride, sugars, like sorbitol, mannitol, or dextran, or other types of polyols such as glycerol.

The inventors surprisingly noticed that glycerol has a non-anticipated but advantageous additional role in the formulation by acting as a co-solvent/co-surfactant improving the physical state of the microsuspension. As a result, glycerol acts as a stabilizer of the microsuspension by reducing the sedimentation rate of the microparticles. Hence, in a preferred embodiment, the ophthalmic composition of the disclosure comprises glycerol.

In certain embodiments with everolimus as the mTOR inhibitor, the amount of glycerol in the aqueous eye drop lipid microsuspension is from 0.1% to 8% w/w, in particular 0.5 to 6% w/w, more particularly about 1% to about 3% w/w, and the amount of everolimus is between 0.01% and 0.1% w/w. In other embodiments, the amount of glycerol in the aqueous eye drop lipid microsuspension may be about 1% and 2.5% w/w, in particular in combination with an amount of about 0.01% to about 0.1% of everolimus, for example about 0.05% w/w of everolimus, in order to adapt the osmolarity of the formulation to about 250 to about 350 mOsmol/kg, preferably about 300 mOsm/kg. More details are also provided in Example 13.

### Polymer and Stabilizing Agent

The ophthalmic composition of the disclosure may further comprise one or more polymer and/or stabilizing agent.

In particular, said polymer or stabilizing agent may be a water-soluble polymer. In specific embodiments, said polymer may be a viscosity enhancing polymer. The term “viscosity enhancing polymer” is intended to mean a polymer that increases the viscosity of a liquid. The increase of viscosity may result in an enhanced physical stability of the composition. As such, the composition is less prone to sedimentation of the solid complex when it comprises a polymer. The polymer may thus be also considered as a stabilizing agent.

In particular, the polymer or stabilizing agent may be a surface active polymer. The term “surface active polymer” is intended to mean a polymer that exhibits surfactant properties. Surface active polymers may, for example, comprise hydrophobic chains grafted to a hydrophilic backbone polymer; hydrophilic chains grafted to a hydrophobic backbone; or alternating hydrophilic and hydrophobic segments. The first two types are called graft copolymers and the third type is named block copolymer.

In one embodiment, the ophthalmic composition of the disclosure comprises a polymer or stabilizing agent selected from the group consisting of a polyoxyethylene fatty acid ester; a polyoxyethylene alkylphenyl ether; a polyoxyethylene alkyl ether; a cellulose derivative such as alkyl cellulose, hydroxyalkyl cellulose and hydroxyalkyl alkylcellulose; a carboxyvinyl polymer such as a carbomer, for example Carbopol® 971 and Carbopol® 974; a polyvinyl polymer; a polyvinyl alcohol; a polyvinylpyrrolidone; a copolymer of polyoxypropylene and polyoxyethylene; tyloxapol; and combinations thereof.

Examples of suitable polymers or stabilizing agent include, but are not limited to, polyethylene glycol monostearate, polyethylene glycol distearate, hydroxypropyl methylcellulose, hydroxypropylcellulose, polyvinylpyrrolidone, polyoxyethylene lauryl ether, polyoxyethylene octyldodecyl ether, polyoxyethylene stearyl ether, polyoxyethylene myristyl ether, polyoxyethylene oleyl ether, sorbitan esters, polyoxyethylene hexadecyl ether (e.g., cetomacrogol 1000), polyoxyethylene castor oil derivatives (e.g., polyoxyl 35 castor oil: Kolliphor® EL, polyoxyl 40 hydrogenated castor oil: Kolliphor® RH40, polyoxyl 15 hydroxystearate: Kolliphor® HS15-BASF), polyoxyethylene sorbitan fatty acid esters (e.g., Tween® 20 and Tween® 80 (ICI Specialty Chemicals)); polyethylene glycols (e.g., Carbowax™ 3550 and 934 (Union Carbide)), polyoxyethylene stearates, carboxymethylcellulose calcium, carboxymethylcellulose sodium, methylcellulose, hydroxyethylcellulose, hydroxypropyl methylcellulose, cellulose, polyvinyl alcohol (PVA), poloxamers (e.g., Pluronics® F68 and FI08, which are block copolymers of ethylene oxide and propylene oxide); poloxamines (e.g., Tetronic® 908, also known as Poloxamine 908, which is a tetrafunctional block copolymer derived from sequential addition of propylene oxide and ethylene oxide to ethylenediamine (BASF Wyandotte Corporation, Parsippany, N.J.)); Tetronic® 1508 (T-1508) (BASF Wyandotte Corporation), Tritons X-200, which is an alkyl aryl polyether sulfonate (Rohm and Haas); PEG-derivatized phospholipid, PEG-derivatized cholesterol, PEG-derivatized cholesterol derivative, PEG-derivatized vitamin A, PEG-derivatized vitamin E, random copolymers of vinyl pyrrolidone and vinyl acetate, combinations thereof and the like.

More particularly, the copolymer of polyoxypropylene and polyoxyethylene may be a triblock copolymer comprising a hydrophilic block-hydrophobic block-hydrophilic block configuration.

Especially useful polymers stabilizing agent stabilizers are poloxamers. Poloxamers can include any type of poloxamer known in the art. Poloxamers include poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate and poloxamer 182 dibenzoate. Poloxamers are also referred to by their trade name Pluronic such as Pluronic 10R5, Pluronic 17R2, Pluronic 17R4, Pluronic 25R2, Pluronic 25R4, Pluronic 31R1, Pluronic F 108 Cast Solid Surfacta, Pluronic F 108 NF, Pluronic F 108 Pastille, Pluronic F 108NF Prill Poloxamer 338, Pluronic F 127, Pluronic F 127 NF, Pluronic F 127 NF 500 BHT Prill, Pluronic F 127 NF Prill Poloxamer 407, Pluronic F 38, Pluronic F 38 Pastille, Pluronic F 68, Pluronic F 68 Pastille, Pluronic F 68 LF Pastille, Pluronic F 68 NF, Pluronic F 68 NF Prill Poloxamer 188, Pluronic F 77, Pluronic F 77 Micropastille, Pluronic F 87, Pluronic F 87 NF, Pluronic F 87 NF Prill Poloxamer 237, Pluronic F 88, Pluronic F 88 Pastille, Pluronic F 98, Pluronic L 10, Pluronic L 101, Pluronic L 121, Pluronic L 31, Pluronic L 35, Pluronic L 43, Pluronic L 44 NF Poloxamer 124, Pluronic L 61, Pluronic L 62, Pluronic L 62 LF, Pluronic L 62D, Pluronic L 64, Pluronic L 81, Pluronic L 92, Pluronic L44 NF INH surfactant Poloxamer 124 View, Pluronic N 3, Pluronic P 103, Pluronic P 104, Pluronic P 105, Pluronic P 123 Surfactant, Pluronic P 65, Pluronic P 84, Pluronic P 85, combinations thereof and the like.

A preferred polymer for use in the composition of the disclosure is poloxamer 407.

The amount of polymer, e.g., poloxamer 407 in the composition of the disclosure may be 0.1% to 5.0% (w/w). In preferred embodiment, the polymer, is present at an amount of about 0.4% to about 1.2%, more particularly about 0.8%, by weight of polymer based on the weight of the composition.

In specific embodiments, the ophthalmic composition comprises poloxamer 407 as a preferred polymer, and/or polyoxyl 15 hydroxystearate as a stabilizing agent, and preferably, the composition comprises a mixture of poloxamer 407 and polyoxyl 15 hydroxystearate, for example in the ratio 0.8:1, respectively.

### Antioxidant and Complexing Agent

One of the known degradation pathways of mTOR inhibitor macrolides is oxidation and, thus, the ophthalmic composition of the disclosure may include one or more antioxidant agent or complexing agent.

Suitable examples of a complexing agent include without limitation EDTA (ethylenediaminetetraacetic acid). The amount of complexing agent in the composition of the disclosure may be 0 to about 0.1%, in particular 0.001 to 0.07%, more particularly 0.03 to 0.07%, even more particularly about 0.05%, by weight of complexing agent based on the weight of the composition.

Suitable examples of antioxidant include without limitation BHA (butylated hydroxyanisol) and BHT (butylated hydroxytoluene), as well as sulfur derivatives, like sodium thiosulfate, sodium bisulfite, sodium metabisulfite, thiourea, alpha-tocopherol and vitamin E TPGS.

The amount of antioxidant in the composition of the disclosure may be 0 to about 1%, in particular 0.05 to 0.5%, by weight of antioxidant based on the weight of the composition.

In specific embodiments, the composition includes at least one complexing agent and no antioxidant. In specific embodiments, the composition includes at least one complexing agent, e.g. EDTA, and at least one antioxidant, for example selected from the group consisting of BHA (butylated hydroxyanisol) and BHT (butylated hydroxytoluene), as well as sulfur derivatives, like sodium thiosulfate, sodium bisulfite, sodium metabisulfite, thiourea, alpha-tocopherol and vitamin E TPGS.

In specific embodiments, the composition does not include any complexing agent but include at least one antioxidant, for example selected from the group consisting of BHA (butylated hydroxyanisol) and BHT (butylated hydroxytoluene), as well as sulfur derivatives, like sodium thiosulfate, sodium bisulfite, sodium metabisulfite, thiourea, alpha-tocopherol and vitamin E TPGS.

### pH of the Composition

In specific embodiments, in particular when the active pharmaceutical ingredient is everolimus, the pH of the composition is maintained between 5.0 and 6.0, preferably between 5.2 and 5.4, for example about 5.3.

Preferred Aqueous Lipid Microsuspensions with Everolimus

In a specific embodiment, the ophthalmic composition is an aqueous eye drop lipid microsuspension comprising at least:
0.01 to 0.1% of everolimus, for example about 0.05%;
α-cyclodextrin; and
castor oil;
wherein the oil and α-cyclodextrin form a microparticle in an aqueous vehicle and the microparticle have a diameter of about 1 μm to 40 μm, more preferably of about 1 μm to 10 μm.

In a preferred embodiment, the ophthalmic composition is an aqueous eye drop lipid microsuspension comprising at least:
0.01 to 0.1% of everolimus, for example about 0.05%;
3 to 5% of α-cyclodextrin, for example about 4.0%; and
2 to 4% of castor oil, for example about 3.0%;
wherein the % are % by weight based on the weight of the composition.

In a further preferred embodiment, the ophthalmic composition is an aqueous eye drop lipid microsuspension comprising at least:
0.01 to 0.1% of everolimus, for example about 0.05%;
3 to 5% of α-cyclodextrin, for example about 4.0%; and
2 to 4% of castor oil, for example about 3.0%; and
1 to 3% of glycerol, for example about 2.0%;
wherein the % are % by weight based on the weight of the composition.

For example, the ophthalmic composition is an aqueous eye drop lipid microsuspension comprising at least:
0.01 to 0.1% of everolimus, for example about 0.05%;
3.5 to 5.5% of α-cyclodextrin, for example about 4.0%; and
2 to 4% of castor oil, for example about 3.0%;
1 to 3% of glycerol, for example about 2.0%;
0.5 to 2% of a stabilizing agent, for example polyoxyl 15 hydroxystearate, typically about 1.0% of polyoxyl 15 hydroxystearate;
0.4 to 1.2% of a polymer, for example poloxamer 407, typically about 0.8% of Poloxamer 407;
0 to 0.07% of a complexing agent, for example disodium edetate dehydrate (EDTA), typically about 0.05% of disodium edetate dehydrate (EDTA);
Optionally, an antioxidant;
wherein the % are % by weight based on the weight of the composition.

### Methods of Preparing the Aqueous Eye Drop Lipid Microsuspension of the Disclosure

The compositions of the disclosure are obtainable by, or obtained by the following methods:

In a specific embodiment, a method of preparing an ophthalmic microsuspension comprises the steps of:
a) preparing an aqueous composition, A by dissolving cyclodextrin in purified water;
b) preparing an oil phase composition B comprising a mTOR inhibitor; preferably said oil being castor oil,
c) optionally, sterilizing the composition B by filtration;
d) adding the oil phase composition B to the aqueous composition A to obtain a mixture C;
e) homogenizing the mixture C, preferably with a high-energy homogenizer to obtain a microsuspension; and,
f) optionally, adding a concentrated aqueous solution of a polymer in the microsuspension.

The step a) of preparing the aqueous composition A includes dissolving cyclodextrin, preferably α-cyclodextrin to obtain an aqueous α-cyclodextrin solution. In specific embodiments, a complexing agent, such as EDTA, and/or a tonicity agent, such as glycerol, are added in composition A.

The composition A thus obtained with dissolved cyclodextrin (preferably α-cyclodextrin) and optionally, EDTA and/or glycerol, in the appropriate amounts may then be autoclaved for sterilization purposes (for example 121 °C for 20 minutes).

Concentrated aqueous solution of a stabilizing agent such as polyoxyl 15 hydroxystearate may also be added in the autoclaved composition A. The aqueous solution of the stabilizing agent can be sterilized by autoclave with the phase A, but preferably, it can be sterilized separately by filtration.

The step b) of preparing the oil phase composition B includes dissolving the mTOR inhibitor, e.g. everolimus, typically by heating at a temperature up to 25 °C to 40 °C for about 10 minutes, preferably between 25 °C to 35 °C. The oil solution with the active pharmaceutical ingredient may then be sterilized by filtration prior to introduction to the aqueous composition A.

Both the aqueous composition A as prepared above and the oil phase B containing the mTOR inhibitor as prepared above are then gently mixed at step d) to obtain a mixture C.

The mixture C is then homogenized, for example with a high-energy homogenizer to obtain the microsuspension. As used herein, the term “high energy homogenizer” refers to means for homogenizing under speed and time conditions suitable to obtain a microsuspension with microparticles preferably with a D50 diameter below 40 μm, preferably below 10 μm. The high-energy homogenizer can, for example, be any high-pressure homogenizer leveraging different settings regarding time and pressure but driving to equivalent results regarding the composition quality.

In specific embodiments, a concentrated solution of a polymer, such as poloxamer 407, may be then added to the microsuspension.

In specific embodiments, the pH may be adjusted to the desired pH, preferably at pH 5.3±0.1 using e.g. concentrated hydrochloric acid solution and/or sodium hydroxide solution. Finally, if required, the weight of the composition can be adjusted with water.

Hence, in more specific embodiments, methods for preparing an aqueous eye drop lipid microsuspension as herein disclosed comprise the steps of:
a) preparing a composition A by dissolving cyclodextrin, preferably α-cyclodextrin, in purified water;
b) adding at least one tonicity agent, for example, glycerol, and at least one antioxidant or complexing agent to the composition A;
c) mixing the composition of step b) and autoclaving, for example, at a temperature of 121 °C for a time t of 20 minutes;
d) adding a sterile aqueous solution of concentrated polymer, such as polyoxyl 15 hydroxystearate, to the sterile composition of step c);
e) dissolving the mTOR inhibitor, preferably everolimus, in an oil, for example castor oil, for example at a temperature comprised between 25 °C and 45 °C, in particular between 25 °C and 35 °C for about 10 minutes to obtain a composition B;
f) adding the composition B sterilized by a filtration, to the composition of step d) to obtain a mixture C;
g) homogenizing the mixture C to obtain a microsuspension;
h) adjusting the pH of the microsuspension to the desired pH, preferably at pH 5.3±0.1, for example by adding a hydrochloric acid solution and/or a sodium hydroxide solution; and,
i) If required, adjusting the final volume or weight of the composition by adding appropriate amount of water.

Step e) may be performed at any time, from prior to step a) until completion of step d).

The homogenization step may be performed using suitable homogenization techniques in order to obtain a microsuspension with appropriate size of microparticles, preferably microparticles with D50 diameter ranging from 1 to 40 μm, more preferably from 1 to 10 μm. High-energy homogenizing parameters are to be fine-tuned specifically depending on the leveraged mixing technology, the formulation composition, the manufacturing batch size and the design of the compounding equipment. Example 17 describes the high-shear homogenization parameters for a typical 100 g composition made in a 125 mL container, including both 3.6% of α-cyclodextrin (dry base) and 1% of castor oil using a an Ultra-Turrax high-shear homogenizer (i.e; 8,000 RPM/10 minutes). In Example 19, a 260 g batch size formulation is made in a 280 mL container, leveraging a formulation including 3% castor oil and 4.0% α-cyclodextrin, leveraging the same Ultra-Turrax equipment with 12,000 RPM/10 minutes settings. Example 23 describes an optimized high-shear homogenization process for a composition including 3.0% castor oil and 4.0% α-cyclodextrin with a high-shear L5M Silverson homogenizer using the “square hole screen” probe (Silverson reference: 7250-HQ0005) operated at 9,000 RPM for 9 minutes.

Further details or examples of manufacturing are provided in the Examples.

### Use of the Aqueous Lipid Microsuspensions

mTOR inhibitor with macrolide ring structure possess both immunosuppressive and anti-inflammatory activity. Those two pharmacological activities are broadly involved in ophthalmology diseases in both front and back of the eye.

Front-of-the-eye or anterior segment conditions are related to physiological disorders at the level of the ocular surface, including the cornea and sclera tissues. Back-of-the-eye or posterior segment conditions are related to physiological disorders located inside the ocular globe and usually more specifically located at the retina tissue level.

Examples of ocular conditions that can be addressed by mTOR inhibitor macrolides are thus all inflammatory diseases of the ocular surface such as dry eye disease (DED), meibomian gland dysfunctions and any ocular Keratitis.

This class of drugs can be of particular interest for addressing either fibrotic or neuro-degenerative diseases of both the anterior and posterior segments.

Hence, the composition of the present disclosure, in particular the preferred aqueous eye drop lipid microsuspensions as disclosed herein, may be used for the treatment of diseases selected from the group consisting of allergic or atopic conjunctivitis (i.e; vernal conjunctivitis), rejection after corneal transplantation, dry eye disease (DED) and meibomian gland dysfunctions.

The composition of the present disclosure, in particular the preferred aqueous eye drop lipid microsuspensions as disclosed herein, is also useful in the treatment or prevention of a corneal endothelial disorder, for instance selected from the group consisting of a corneal endothelial disorder, for instance selected from a corneal dystrophy, in particular Fuchs' endothelial corneal dystrophy, posterior polymorphous dystrophy, congenital hereditary endothelial dystrophy, corneal endotheliitis, cytomegalovirus corneal endotheliitis, idiopathic corneal endothelial disorder, post-ophthalmic surgery disorder, in particular post-ophthalmic laser surgery disorder, trauma and aging.

As used herein, the terms “treating” or “treatment” of a disease, disorder, or syndrome, includes (i) preventing the disease, disorder, or syndrome for occurring in a subject, i.e. causing one or more clinical symptoms of the disease, disorder or syndrome not to develop in an animal that may be exposed or predisposed to the disease, disorder or syndrome, but does not yet experience or display one or more symptoms of the disease, disorder or syndrome; (ii) inhibiting the disease, disorder or syndrome, or at least one or more symptoms of the disease, disorder, or syndrome, or (iii) relieving the disease, disorder or syndrome or one or more of their symptoms, i.e. causing regression of the disease, disorder or syndrome.

The anti-inflammatory property is also of particular interest. The antifibrotic properties of some specific mTOR inhibitor macrolides like everolimus, can be further leveraged in ophthalmic related cancer diseases. This antifibrotic activity appears of specific interest in the filtering surgery of glaucoma for preventing scar formation too. The composition of the disclosure may also be used in trabeculectomy, typically as a post-surgical treatment. The composition can be used in trabeculectomy to prevent the ostium to be blocked during post-surgery wounding process. The antifibrotic properties of mTOR inhibitors such as everolimus can also be leveraged in the treatment of pterygium. In specific embodiments, the disclosure thus relates to the use of the composition in the treatment of pterygium. The addition of both the antifibrotic and the immunosuppressive properties of everolimus makes this specific compound a particularly good candidate for reducing the risk of corneal graft rejection after ocular surgery. Hence, the composition of the present disclosure, in particular the preferred aqueous eye drop lipid microsuspensions as disclosed herein may be used for the prevention of corneal graft rejection in a subject in need thereof, in particular for high risk corneal transplants. In this specific application, everolimus is expected to be superior to tacrolimus which is used in some topical ophthalmic formulations for years.

As used herein, the term “subject” refers to a mammal, preferably a human subject.

The composition of the present disclosure is also useful for treating uveitis in a subject in need thereof, as a topical ophthalmic formulation would be preferred for everolimus administration, in particular for improving patient treatment adherence by being more patient friendly and less risky as a medical practice.

The present disclosure also covers the use of the ophthalmic composition of the disclosure as an eye drop solution.

In various embodiments, the composition of the disclosure, in particular, any of the preferred aqueous eye drop lipid microsuspensions as disclosed in the above sections, is administered in a therapeutically effective amount. As used herein, the term “therapeutically effective amount” refers to any amount which, as compared to a corresponding subject who has not received such amount, which results in improved treatment, healing, prevention or amelioration of a disease or disorder, or a decrease in the rate of advancement of a disease or disorder, or which includes amounts effective to enhance normal physiological function.

In some embodiments, the compositions herein are used in methods for treating or reducing inflammation in the eye in a subject in need thereof, said method comprising topically administering an effective amount of a composition as disclosed herein in the eye or the eyelid of said subject.

For example, in a preferred embodiment, the composition of the disclosure, typically the preferred aqueous eye drop lipid microsuspension with everolimus as disclosed in the previous section, are used in the prevention of rejection of corneal transplantation. In such preferred embodiment, the microsuspension may be topically administered to the eye in an amount of 1 drop, one to 3 times per day for 2 to at least 12 months, or more, starting from 4 weeks after the surgical operation. In other embodiments, the microsuspension is topically administered to the eye in an amount of 1 drop, one to six times per day.

In other embodiments, the composition of the disclosure, typically the preferred aqueous eye drop lipid microsuspension with everolimus as disclosed in the previous section, are used in the treatment of dry eye disease or meibomian gland dysfunctions possibly including blepharitis. In such embodiment, the microsuspension is topically administered to either the eye or the eyelid in an amount of 1 drop, one to 3 times per day for 2 to at least 12 months, or more.

In some embodiments, the compositions herein are used in methods for treating or reducing cellular immunity in the eye in a subject in need thereof, said method comprising topically administering an effective amount of a composition as disclosed herein in the eye or the eyelid of said subject.

The composition of the invention is also suitable for treating skin diseases, including inflammatory or immunological related skin disorders. For instance, the composition can be administered to treat psoriasis, atopic dermatitis and/or other skin diseases sensitive to anti-inflammatory drugs and/or immunosuppressor drugs. In such applications, the composition is administered topically to the skin of a subject.

### EXAMPLES

### Example 1: Methods for Characterizing the Microsuspension

### Determining Diameter of a Microparticle in the Microsuspensions

The diameter of a particle, such as a microparticle of mTOR inhibitor in oil and cyclodextrin/lipid complexes, can correspond to the D50 diameter of the particle. Diameter D50 is also known as the median diameter or the medium value of the particle size distribution. Diameter D50 corresponds to the value of the particle diameter at 50% in the cumulative distribution. For example, if D50 is 5 μm, then 50% of the particles in the sample are larger than 5 μm, and 50% smaller than 5 μm. Diameter D50 is usually used to represent the particle size of a group of particles.

The diameter and/or size of a particle can be measured according to any method known to those of ordinary skill in the art. For example, the diameter D50 is measured by laser diffraction particle size analysis. Generally, there are a limited number of techniques for measuring/evaluating cyclodextrin/drug particle or complex diameter and/or size. In particular, persons of ordinary skill in this field know that the physical properties (e.g. particle size, diameter, average diameter, mean particle size, etc.) are typically evaluated/measured using such limited, typical known techniques.

For example, such known techniques are described in Int. J. Pharm. 493 (2015), 86-95, cited above in paragraph [00076], which is incorporated by reference herein in its entirety. In addition, such limited, known measurement/evaluation techniques were known in the art as evidenced by other technical references such as, for example, European Pharmacopoeia (2.9.31 Particle size analysis by laser diffraction, January 2010), and Saurabh Bhatia, Nanoparticles types, classification, characterization, fabrication methods and drug delivery applications, Chapter 2, Natural Polymer Drug Delivery Systems, PP. 33-94, Springer, 2016, which are also incorporated by reference herein in their entireties.

In specific embodiments, the particle size is measured by laser diffraction particle size analysis according to Pharm. Eur. 2.9.31 with the following parameters:
System: Malvern Mastersizer 3000 with hydro MV disperser
Fraunhofer approximation
Dispersant: water
Refractive index of the dispersant: 1.33
Time of measurement: 1 second
Time of measurement of the background: 10 second
Stirrer speed: 3500 rpm
Obscuration range: 1-20%
Model: standard
Sample preparation: Homogenize the eye drops by shaking
Sample size: addition of 0.5 ml eye drop to the disperser
Cleaning: rinsing twice with the dispersant (water) and start a measurement, checking that beam strength is less than 120 units in the first channels, and loading a background.

### Determining Stability of the Active Pharmaceutical Ingredient in the Microsuspension

Quantitative determination of everolimus was performed on a reversed phase liquid chromatographic component system (HPLC system) consisting of pump operated at 1.1 mL/min, auto sampler, column compartment operated at 50 °C, UV-Vis detector operated at 210 nm and 275 nm, and Thermo BDS Hypersil® C18, 250×3.0 mm, 5 μm column. Injection volume 10 μL. Operation time 23 minutes. The gradient mobile phase system consisted of A: 2 mM Potassium dihydrogen phosphate, and B: acetonitrile:

TABLE 1

| Time (min) | Mobile phase A (%) | Mobile phase B (%) |
|---|---|---|
| 0 | 50 | 50 |
| 7 | 35 | 65 |
| 10 | 0 | 100 |
| 15 | 50 | 50 |
| 23 | 50 | 50 |

Preparation of standard solution: Accurately weigh and transfer about 25 mg of active pharmaceutical ingredient into a 50 mL volumetric flask, dissolve and fill up to the volume with methanol. Take 1 mL of the stock solution and transfer it into a 10 mL volumetric flask, fill up to the volume with methanol (final conc. 0.05 mg/mL).

Preparation of the test solution: Transfer 1 mL of the microsuspension to a 10 mL volumetric flask and fill up the volume with methanol.

Typically, for assessing the stability of everolimus in a media, the drug-substance assay in the media is performed by HPLC technique at different time points, while different samples from the same original product are stored for a period of time at different storage conditions (i.e. different temperatures).

### Example 2: Everolimus Solubility in Different Media

Excess everolimus was added to each media, the samples were continuously agitated over night at room temperature. The next day the samples were filtered through 0.2 μm PES membrane filters and the concentration of dissolved everolimus determined by HPLC as described in Example 1. The results are displayed in Table 2. Then the visual appearance of the samples was recorded after storage at 70 °C for two days.

TABLE 2
The media composition and solubility of everolimus at room temperature.

| | | | Everolimus |
|---|---|---|---|
| | | | solubility |
| Solubilizer | Brand name | Media | (% w/w) |
| Polysorbate 80 | Tween ® 80 | 10% (w/w) solution in | 1.3 ^{a} |
| | | water | |
| Polyoxyl 40 | Kolliphor ® RH40 | 10% (w/w) solution in | 1.6 ^{a} |
| hydrogenated | | water | |
| castor oil | | | |
| Polyoxyl 35 | Kolliphor ® EL | 10% (w/w) solution in | 1.7 ^{b} |
| castor oil | | water | |
| Polyoxyl 15 | Kolliphor ® HS15 | 10% (w/w) solution in | 1.5 ^{c} |
| hydroxystearate | | water | |
| Polyoxyl 40 | Myrj ™ S40 | 10% (w/w) solution in | 1.3 ^{a} |
| stearate | | water | |
| Polyvinyl | Polyvinyl alcohol | 1.4% (w/w) solution | Below limit of |
| alcohol | 40/88 | in water | detection |
| Phosphatidyl- | Lipoid ® S100 | 5% (w/w) solution in | 1.0 ^{d} |
| choline | | water | |
| PEG 400 | Kollisoly ® | 100% | 19.5 ^{d} |
| | PEG400 | | |
| Poloxamer 407 | Kolliphor ® P407 | 10% (w/w) solution in | 1.3 ^{c} |
| | | water | |

^{a} Precipitation after storage at 70 °C for two days.
^{b} Turned opalescence after storage at 70 °C for two days.
^{c} The solution did not change during storage at 70 °C for two days.
^{d} The solution turned yellow after storage at 70 °C for two days.

### Example 3: a Simple Surfactant Solubilization of Everolimus is not Sufficient for Acceptable Stabilization

Stability of everolimus was determined in aqueous 1% (w/w) solutions of selected surfactants. The surfactant was dissolved in water containing pH 5.0 0.1 M citric/phosphate buffer. These buffer-surfactant solutions were filled in 5 mL clear, sealed glass vials and heated to 70 °C. Then 0.1% (w/w) of everolimus was added at time zero. The everolimus concentration in the solutions was determined at time zero and then after 24 and 72 hours. A simple surfactant solubilization of everolimus did not result in acceptable stabilization of the drug.

TABLE 3
Concentration of everolimus as the percent of the initial concentration. The initial
everolimus concentration was 0.1% (w/w), the media was aqueous
pH 5.0 buffer solution, and the storage temperature was 70 °C.

| | | Everolimus concentration | | |
|---|---|---|---|---|
| | | (% of initial concentration) | | |
| | | 0 | 24 | 72 |
| Solubilizer | Brand name | hour | hours | hours |
| 1% (w/w) Polyoxyl 35 castor oil | Kolliphor ® EL | 100 | 90.7 | 72.7 |
| 1% (w/w) Polyoxyl 15 | Kolliphor ® | 100 | 95.9 | 85.4 |
| hydroxystearate | HS15 | | | |
| 1% (w/w) Phosphatidylcholine | Lipoid ® S100 | 100 | 65.1 | 4.1 |
| 1% (w/w) Poloxamer 407 | Kolliphor ® | 100 | 93.5 | 74.5 |
| | P407 | | | |

### Example 4: Natural γ-Cyclodextrin has the Highest Affinity for Everolimus and α-Cyclodextrin the Lowest Affinity

The phase-solubility of everolimus was determined in pure aqueous cyclodextrin solutions at 25 °C. The cyclodextrin concentration ranged from 0 to 10% (w/w) except that of β-cyclodextrin that ranged from 0 to 1.50% (w/w). Everolimus was added to each cyclodextrin solution in excess, the samples were continuously agitated on a magnetic stirrer for 24 hours. The next day the samples were filtered through 0.45 μm PES syringe filters and the concentration of dissolved everolimus determined by HPLC as described in Example 1.

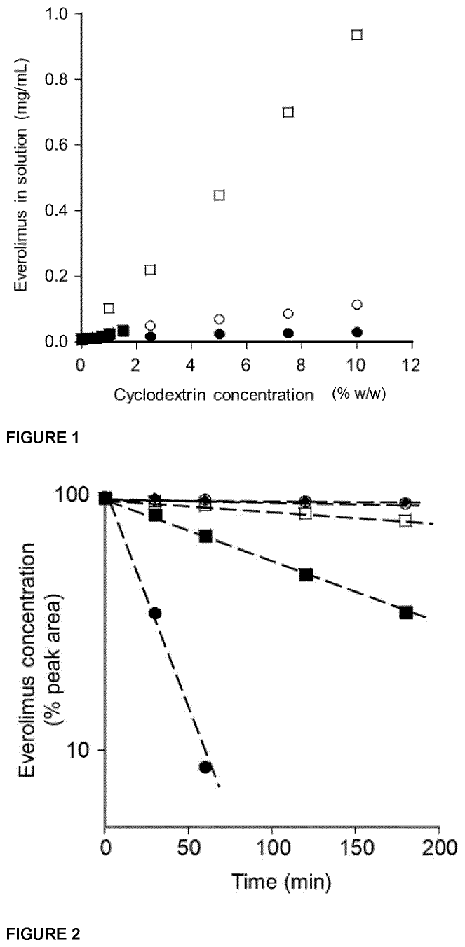

The results are displayed in FIG. 1. The figure shows that the natural γ-cyclodextrin has the highest affinity for everolimus and α-cyclodextrin the lowest affinity. Even though the natural γ-cyclodextrin had the highest affinity for everolimus about 80 mM of γ-cyclodextrin is needed to dissolve 1 mM of everolimus.

### Example 5: Everolimus has Maximum Stability Between pH 4 and 5

4 mg of everolimus was dissolved in 800 μL of acetonitrile, 1.2 mL McIlvaine buffer ranging from pH=4 to pH=8 was added, giving initial everolimus concentration of 2 mg/mL, and the solutions were thermostated at 70 °C. The everolimus content in the samples were quantified as percent peak area of the initial value.

The results in FIG. 2 show that the everolimus degradation follows first-order kinetics in pure aqueous solutions, displaying maximum stability at pH from about 4 to below 6.

### Example 6: A Simple Cyclodextrin Complexation of Everolimus Did not Result in Acceptable Stabilization of the Drug

The chemical stability of everolimus was studied in pure aqueous 15% (w/w) γ-cyclodextrin solution saturated with everolimus. The rate of everolimus degradation was determined at 70 °C and pH about 4.2. Complexation with γ-cyclodextrin resulted in about a three-fold increase in the chemical stability of everolimus compared to the degradation rate in aqueous 40% acetonitrile solution (Example 5). However, the shelf-life (i.e., time for 10% degradation or too) was only 1 day. Accordingly, the estimated too at 25 °C is only 2 to 6 months and only 8 to 24 months at 5 °C. Everolimus degraded via acid catalyzed pathway as well by oxidation. A simple cyclodextrin complexation of everolimus did not result in acceptable stabilization of the drug.

### Example 7: Dissolution of Everolimus in Castor Oil and Medium-Chain Triglycerides

The ability of castor oil and medium-chain triglycerides Ph. Eur. (Kollisolv® MCT 70) to dissolve 5% (w/w) everolimus was determined. 75 mg of everolimus was suspended in both 1.5 mL of castor oil and medium-chain triglycerides. Everolimus dissolved relatively quickly in medium-chain triglycerides, while castor oil required some time to dissolve, likely due to its higher viscosity. The samples were kept under stirring for 24 hours at room temperature. After 24 hours, castor oil had formed a clear everolimus solution while the previously clear medium-chain triglycerides solution had turned into a white viscous gel. The castor oil solution was observed for several days and remained stable during that time. Further studies showed that 7.5% (w/w) everolimus in castor oil formed a stable solution.

### Example 8: Everolimus is More Stable in Microsuspensions with α-Cyclodextrin and Oil

The chemical stability of 0.05% everolimus was evaluated in three castor oil/cyclodextrin suspensions and two reference solutions, one containing 15% (w/w) γ-cyclodextrin and the other containing 1% tyloxapol (a nonionic surface-active polymer with HLB value of 12.9). The suspensions and reference solutions were stored at 60 °C in sealed glass containers and the everolimus concentration determined by HPLC at various time points as described in Example 1. The compositions and the everolimus concentrations at day 0, 1 and 3 are shown in Table 4. β-Cyclodextrin has low aqueous solubility and, thus, only 1% (w/w) β-cyclodextrin suspension was evaluated. The 2% oil/8% α-cyclodextrin suspension displayed the highest everolimus stability. The stability decreased when the oil and α-cyclodextrin concentrations were lowered, presumably due to decreased fraction of everolimus being located well within the oily droplets (or oil microparticles). Solutions without oil were much less effective in protecting everolimus against degradation. However, the physical stability of the cyclodextrin-based suspensions were limited.

TABLE 4
The composition of the lipid suspensions the aqueous reference solutions, and the
concentration of everolimus remaining upon storage at 60 °C.

| Cyclodextrin or | | Everolimus remaining (mg/mL) | | |
|---|---|---|---|---|
| solubilizer | Castor oil | Day 0 | Day 1 | Day 3 |
| 8% (w/w) α-Cyclodextrin | 2% (w/w) | 0.501 | 0.503 | 0.505 |
| 4% (w/w) α-Cyclodextrin | 1% (w/w) | 0.492 | 0.495 | 0.454 |
| 1% (w/w) β-Cyclodextrin | 0.80% (w/w) | 0.504 | 0.500 | 0.478 |
| 15% (w/w) γ-Cyclodextrin | — | 0.444 | 0.417 | 0.345 |
| 1% (w/w) Tyloxapol | — | 0.465 | 0.460 | 0.442 |

### Example 9: Everolimus Stability is not Sufficient in Conventional Emulsion

The chemical stability of 0.05% everolimus was evaluated in a conventional emulsion and compared to a solution including a surfactant. The solution contained 0.1% of everolimus dissolved in water with 1% of Kolliphor® HS15, and a pH 5.0 citric/phosphate 0.1M buffer system. The emulsion contained everolimus (0.05%), castor oil (0.8%), glycerol (2.5%), EDTA (0.05%) and Kolliphor® HS15 (1%). The emulsion was prepared at room temperature by dissolving everolimus in the oil followed by high-speed mixing with a Kolliphor® HS15 aqueous solution. An Ultra-Turax® T-25 Classic homogenizer (Ika-Werke) with the S25KD-18G mixing probe was used in this experiment. The mixing speed starts at 11,000 RPM and is increased up to 24,000 RPM for 2 minutes. Then, a solution containing the remaining other ingredients was added to the previous emulsion and homogenized again with a high-speed homogenizer as previously described. The two samples were then filled into sealed glass vials and stored at 70 °C.

After storage for one week, the everolimus concentration was determined as described in Example 1. The drop of everolimus concentration in the solution was about 35% while the drop of everolimus concentration in the emulsion was about 27%. Even if the stability of everolimus in this emulsion appears to be better than in the aqueous solution, the everolimus stability in the emulsion was not acceptable.

### Example 10: Everolimus is not Stable after Autoclave in Aqueous Solution

The chemical stability of an everolimus aqueous solution upon sterilization by autoclave was evaluated. A stock solution of 15% (w/w) γ-cyclodextrin was prepared without buffer. Excess amount of everolimus, approximately 10 mg/mL, was added to the stock solution. The obtained suspension was gently sonicated for about 5 min, vortexed, and mixed overnight at 25 °C on a magnetic stirrer. The next day the suspension was filtered with a 0.45 μm syringe filter (PES material, from Agilent). A part of the filtered solution is saved for analysis and the other part is filled in a sealed glass vial and exposed to an autoclave cycle (121 °C for 20 minutes). Then the two samples were tested by HPLC according to Example 1. The not autoclaved sample contained 1.4 mg/mL of everolimus at pH 4.9. After autoclaving, the loss of everolimus was 90.8%.

### Example 11: Chemical Stability of Everolimus is Increased in Microsuspensions with Increased Concentration of Oil

Two eye drop formulations were prepared and the chemical stability of everolimus was determined at 5 °C, 25 °C, and 40 °C. The composition of the eye drops is shown in Table 5. The results of the chemical stability study are shown in Table 6. Increasing the castor oil concentration from 1% (Composition A) to 3% (Composition B) resulted in significant enhancement of the chemical stability of everolimus or by about 2.5 to 3-fold less degradation. Samples of both composition A and B are prepared according to example 17.

TABLE 5
The composition of the aqueous eye drops
containing everolimus in lipid microsuspension,
and the results of the stability study (% w/w).

| | Composition A | | Composition B | |
|---|---|---|---|---|
| Everolimus | 0.05% | | 0.05% | |
| Castor oil | 1.0% | | 3.0% | |
| α-Cyclodextrin | 3.6% | | 3.6% | |
| Glycerol | 2.0% | | 2.0% | |
| Kolliphor ® HS15 | 1.0% | | 1.0% | |
| Poloxamer 407 | 0.8% | | 0.8% | |
| Disodium edetate | 0.05% | | 0.05% | |
| dehydrate (EDTA) | | | | |
| Purified water | q.s. 100 | | q.s. 100 | |
| Hydrochloric acid or | q.s. pH 5.3 ± 0.1 | | q.s. pH 5.3 ± 0.1 | |
| Sodium hydroxide | | | | |
| pH | 5.4 | | 5.4 | |
| Viscosity | 4.5 | cP | 7.7 | cP |
| Osmolality | 279 | mOsmol/kg | 287 | mOsmol/kg |
| Particle size Dx(50) | 6.5 | μm | 8.5 | μm |

TABLE 6
Degradation of everolimus in Composition A and B. The
percent everolimus remaining after storage at 5 °C, 25 °C
and 40 °C, when stored in type I glass primary containers.

| | 5 ± 2 °C | 25 °C/60% RH | 40 °C/25% HR |
|---|---|---|---|
| Composition A | | | |
| 0 days | 100% | 100% | 100% |
| 45 days | 99.9% | 99.2% | 95.4% |
| 90 days | 99.8% | 98.2% | 90.9% |
| 180 days | 99.4% | 96.8% | 83.8% |
| Composition B | | | |
| 0 days | 100% | 100% | 100% |
| 45 days | 99.9% | 99.6% | 98.4% |
| 90 days | 99.9% | 99.4% | 96.3% |
| 180 days | 99.6% | 98.5% | 93.6% |

### Example 12: Eye Drop of Vehicle A and B are Well Tolerated in Human

Two eye drop vehicles of identical compositions to those described in Table 5 but without everolimus. Two healthy male volunteers received one drop of Vehicle A in the left eye and 24 hours later one drop of Vehicle B in the left eye. Slightly blurred vision was observed for about 10 seconds after administration of the eye drops. No burning sensation, itching or other side effects were observed. The eye drops were well tolerated.

### Example 13: Effect of Glycerol on the Microsuspension

In a specific experiment, different concentrations of glycerol were included in the formulation in order to study their possible impact. The samples were prepared according to example 17.

The tonicity of the samples increases proportionally to their content of glycerol. Also, it appears that the physical state of the formulation is significantly different in absence of glycerol: Formulation #1, which does not contain any glycerol, has a different physical appearance compared to Formulation #2 and #3. In addition, when exposed to the centrifugation test, the total content of sedimented lipid microparticles, the white product fraction which is at the bottom of the centrifugation tube, is quantitatively about 20% lower compared to the 2 other samples, demonstrating a faster formulation sedimentation. This can be extrapolated to a product with a lower physical stability because this sample without glycerol will sediment faster compared to the other samples, when stored at rest for long term in normal storage conditions.

TABLE 7
Composition and physiochemical characteristics of lipid
microsuspensions containing different amounts of glycerol.

| Ingredients | Formulation 1 | Formulation 2 | Formulation 3 |
|---|---|---|---|
| Glycerol | 0 | 1% | 2.5% |
| Castor oil | 1% | 1% | 1% |
| Kolliphor ® HS15 | 1% | 1% | 1% |
| Poloxamer 407 | 0.8% | 0.8% | 0.8% |
| Alpha-cyclodextrin | 3.6% | 3.6% | 3.6% |
| Everolimus | 0.05% | 0.05% | 0.05% |
| EDTA (Na₂) | 0.05% | 0.05% | 0.05% |
| HCl/NaOH (0.25N) | Q.S. pH 5.3 | Q.S. pH 5.3 | Q.S. pH 5.3 |
| Purified water | Q.S. 100 | Q.S. 100 | Q.S. 100 |
| Tests | | | |
| Sedimentation rate (*) | 35% | 45% | 45% |
| pH | 5.4 | 5.4 | 5.4 |
| Tonicity | 25 mOsmol/kg | 143 mOsmol/kg | 357 mOsmol/kg |
| Particles | | | |
| Dx10 (μm) | 1.3 | 1.3 | 1.2 |
| Dx50 (μm) | 5.7 | 4.9 | 4.4 |
| Dx90(μm) | 18 | 18 | 16 |

(*) Sediment (solid material) from a product sample after centrifugation at 13,500 RPM during 20 min.

### Example 14: Effect of High Shear Mixing Speed at Constant Mixing Time

The mixing speed implemented during the high shear homogenization step of the formulation manufacturing process leveraging the Ultra-Turrax® tool, as described in Example 17, was evaluated. Different samples were prepared and for each of them, the high shear homogenization step has been performed with a specific mixing speed, covering a range from 4,000 RPM up to 20,000 RPM for 10 minutes.

The results show physical appearance improvement of the formulation, usually driving to a formulation with an improved sedimentation rate and a decrease of the particle sizes. The higher the mixing speed, lower are the particles sizes, with an improved particle size distribution profile (more homogenous).

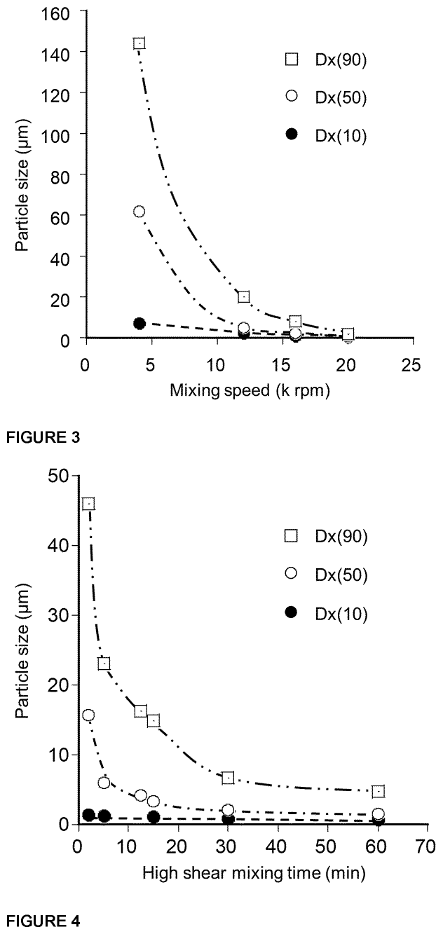

FIG. 3 shows the particle sizes evolution across different high shear mixing speeds for a formulation containing 1% oil (see Composition A in Example 11). If at laboratory scale an ideal high shear mixing speed should be higher than either 8,000 RPM (for 1% oil) or 12,000 RPM (for 3% oil), these speeds can be further increased and optimized at larger industrial scale batch manufacturing and depending on either the geometry of the leveraged high shear homogenizers (e.g., IKA, Silverson, or other homogenizer brands), or the mixing technology: high-shear vs. high-pressure homogenizer (i.e., IKA, Hommac, GEA, or other homogenizer brands).

### Example 15: Effect of High Shear Mixing Time at Constant Mixing Speed

The mixing time implemented during the high shear homogenization step of the formulation manufacturing process leveraging the Ultra-Turrax® tool, as described in Example 17, was evaluated. Different samples were prepared and for each of them, the high shear homogenization step was performed with a specific mixing duration, covering a range from 4 minutes up to 20 minutes at the constant mixing speed of 8,000 rpm. The results show physical appearance improvement of the formulation, usually driving to a formulation with an improved sedimentation rate and a decrease of the particle sizes. Higher is the mixing duration, lower are the particles sizes, with an improved particle size distribution profile (more homogenous). The plot in FIG. 4 shows the particle sizes evolution across different high shear mixing times at 8,000 RPM for a formulation containing 1% oil (see composition A from Example 11). If at laboratory scale an ideal high shear mixing duration should be higher than either 10 minutes (for both 1% and 3% oil), this duration can be further increased and optimized at larger industrial scale batch manufacturing and depending on either the geometry of the leveraged high shear homogenizers (e.g., IKA, Silverson, or other homogenizer brands), or the mixing technology: high-shear vs. high-pressure homogenizer (i.e., IKA, Hommac, GEA, or other homogenizer brands).

### Example 16: Maximum Stability of Everolimus at pH Between 5.2 and 5.4

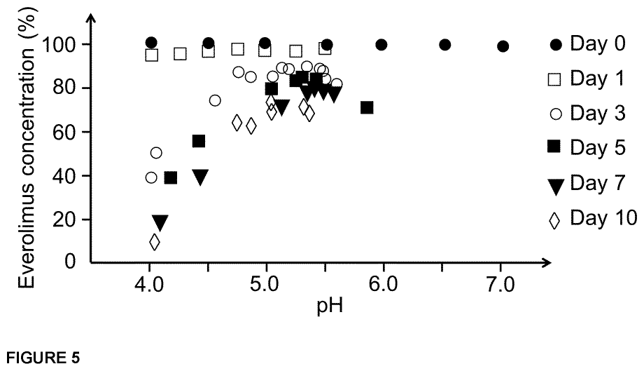

Everolimus is, like other macrolide mTOR inhibitors, sensitive to hydrolysis. When solubilized in aqueous solution, everolimus can be degraded rapidly. This degradation is influenced by the pH of the aqueous environment. The pH of best stability was determined by solubilizing everolimus (0.025% w/w) in aqueous 14% (w/w) 2-hydroxypropyl-β-cyclodextrin solution containing 0.1% (w/w) tyloxapol. The pH of the final solution was adjusted with concentrated hydrochloric acid solution and/or concentrated sodium hydroxide solution. The final everolimus solutions were stored in sealed glass containers at 40 °C and analyzed at various time points as described in Example 1. The results shown in FIG. 5 below indicate that in aqueous solutions everolimus has maximum stability at pH between 5.2 and 5.4.

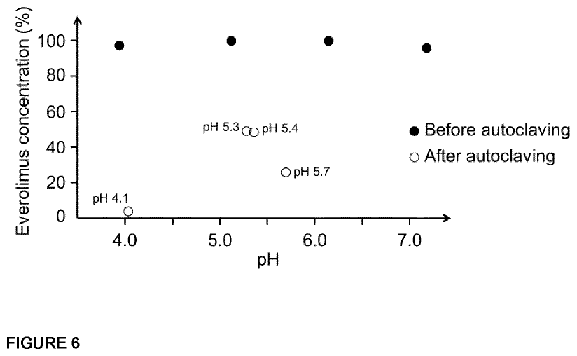

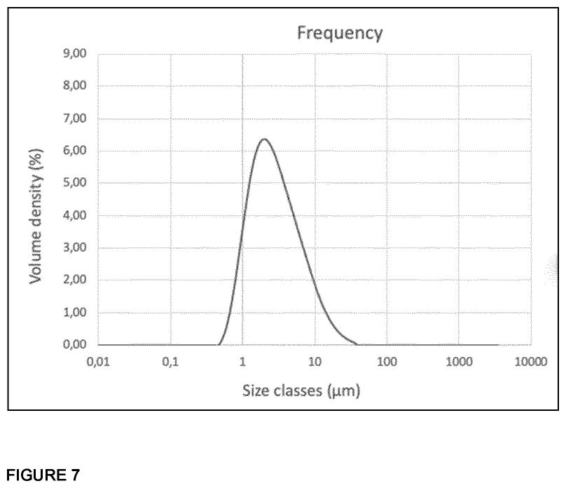

The solutions were also heated in sealed glass contains in an autoclave at 121 °C for 20 minutes and the pH and concentration of everolimus determined before and after heating (FIG. 6). Maximum stability was observed at pH between 5.3 and 5.4.

### Example 17: Mixing Order of the Ingredients of the Lipid Microsuspension

Following is a general procedure for preparation of a non-sterile lipid microsuspension at laboratory scale and for a total batch size of 100 g:

First, appropriate quantity of castor oil containing the dissolved drug-substance (i.e., everolimus) is transferred into a glass container. Second, under stirring with a magnetic stirrer aqueous α-cyclodextrin solution is added to the container. Third, aqueous solution containing EDTA and glycerol is added. Fourth, aqueous solution of Kolliphor HS15 is added. Mixing is continued for about 10 minutes before the stirring bar is removed and the suspension formed mixed with a high-shear homogenizer probe (Ultra-Turrax® T25 homogenizer, IKA-Werke, Germany, with probe IKA-S-25N-18G) operated at 8,000 RPM for about 10 minutes. Then aqueous poloxamer 407 solution is added and mixed carefully with magnetic stirrer for about 30 minutes. The pH is adjusted to 5.3±0.1 with concentrated hydrochloric acid solution and/or sodium hydroxide solution. Finally, if needed, purified water is added to adjust the final weight of the sample to 100%.

In this example, poloxamer 407 should preferably not be introduced before the high shear homogenization because it will generate a significant amount of foam that prevents sufficient formulation homogenization. This will affect both the rheological properties and the particle size distribution of the formulation. On the other hand, Kolliphor HS15 should preferably be introduced before the high shear homogenization step. If it is done after homogenization the lipid microsuspension may not have the targeted physical properties. The oil shall be introduced before the high shear homogenization.

### Example 18: Sterilization of the Microsuspension at Industrial Manufacturing Scale

Topical eyedrops must be sterile drug-products. This means that their manufacturing process must include a sterilization step to provide sterile products in a primary container enabling to keep the integrity of the liquid formulation up to the moment of its used by the patient.

Usually, sterile liquids are sterilized by heat leveraging a terminal sterilization of the solution by autoclave (e.g., 121 °C for 20 minutes). Previous examples demonstrate that everolimus, like the other macrolide mTOR inhibitors, is strongly heat sensitive and degrades during autoclaving. As a result, one of the challenges of manufacturing lipid microsuspensions containing a macrolide mTOR inhibitor is sterilization of the final eye drops. One of the advantages of the formulation is to allow to produce a sterile drug formulation without exposing everolimus, or other mTOR macrolides, to heat sterilization. For doing that, an aseptic manufacturing process including both autoclave and aseptic filtration steps is leveraged as described below.

On an industrial scale, a double jacket stainless steel tank is used for the manufacturing of the formulation. The tank is equipped with a conventional mixing system either by stirring blade or magnetic stirrer. The tank is also equipped with an embedded high-shear homogenizing tool (either high-shear or high-pressure). A part of purified water is introduced into the industrial stainless-steel tank and α-cyclodextrin is dissolved in the water. Then, both EDTA and glycerol are added. The tank is then closed, and the solution autoclaved (121 °C for 20 minutes). This autoclave step allows sterilization of all the parts of the manufacturing equipment that are in direct contact with the product. A concentrated aqueous solution of Kolliphor HS15 is prepared in parallel and introduced into the tank after the autoclaving, when the temperature has been reduced to 25 °C, through a PES filter of 0.2 μm porosity. A PES Supor® filter (Pall, USA), or equivalent, can be leveraged. In a next step, the active pharmaceutical ingredient, that is everolimus, is dissolved in the oil in a separate stainless-steel container that can be slightly heated up to a 25 °C-35 °C temperature range for about 10 minutes, facilitating the dissolution of the drug. The oil solution, still at a temperature of at least 30 °C, is then added to the tank through another PES filter of 0.2 μm porosity. The 0.2 μm filtration steps allow sterilization of the liquids before their introduction into the tank containing the sterile bulk solution. Then the high shear homogenizer, which is installed in the tank and previously sterilized during the autoclave phase, is triggered and the microsuspension is formed. Then, a concentrated aqueous solution of poloxamer 407 is introduced into the tank through another 0.2 μm PES filter. The pH of the microsuspension is adjusted to 5.3±0.1 with concentrated hydrochloric acid solution and/or sodium hydroxide solution, introduced into the tank through the 0.2 μm PES filter. Finally, the weight of the aqueous microsuspension is adjusted by introducing the remaining amount of purified water through the 0.2 μm PES filter. The final bulk formulation is left under moderate mixing in the sterile tank for at least one hour at a temperature not exceeding 25 °C. The tank containing the sterile formulation is finally aseptically connected to a blow-fill-seal machine enabling to fill single-dose units eyedrops containers (i.e., Rommelag Kunststoff-Maschinen Vertriebsgesellschaft mbH, Germany, or Weiler Engineering, Inc, USA, or equivalent).

### Example 19: Aqueous Everolimus Lipid Microsuspension Containing 3% Castor Oil

Composition of an aqueous everolimus lipid microsuspension containing 3% castor oil is shown in Table 8. The lipid microsuspension was prepared as described in example 17 but with the high shear homogenization step performed at 12,000 rpm for 10 minutes (Ultra-Turrax®).

TABLE 8
Composition of an aqueous 0.05% everolimus lipid
microsuspension containing 3% castor oil.

| Ingredient | Concentration | |
|---|---|---|
| Everolimus | 0.05% | |
| Castor oil | 3.0% | |
| α-Cyclodextrin | 4.0% | |
| Glycerol | 2.0% | |
| Kolliphor ® HS15 | 1.0% | |
| Poloxamer 407 | 0.8% | |
| Disodium edetate dehydrate (EDTA) | 0.05% | |
| Purified water | q.s. 100 | |
| Hydrochloric acid or Sodium hydroxide | q.s. pH 5.3 ± 0.1 | |
| pH | 5.3 | |
| Viscosity | 11 | cP |
| Osmolality | 284 | mOsmol/kg |
| Particle size Dx(50) | 5.0 | μm |

### Example 20: The Eye Drop Vehicle is Suitable for a Wide Variety of Drugs

Eye drops containing a given drug (everolimus, tacrolimus, sirolimus, cyclosporin A, loteprednol etabonate, or dexamethasone) were prepared in a 250 mL glass bottle as follows (all w/w %): 8 g of castor oil containing the drug (0.05 to 0.1%) was added to 133.4 g of an aqueous stock solution containing 8.0% α-cyclodextrin, 4.0% glycerol and 0.1% EDTA, followed by 58.7 g of an aqueous stock solution containing 4.55% Kolliphor® HS15 and 0.3595% Vitamin E-TPGS. The mixture obtained was homogenized for 10 minutes at 12,000 rpm using Ultra-Turrax® high shear homogenizer with S25N-18G probe at 12,000 rpm for 10 minutes. Then 66.7 g of an aqueous 3.217% poloxamer 407 solution was added under stirring. The lipid microsuspension formed was stirred by magnetic stirrer for 30 minutes at room temperature. Finally, the pH was adjusted by addition of hydrochloric acid and/or sodium hydroxide. The final composition of the aqueous lipid microsuspension vehicle was as follows: Castor oil 3.00%, EDTA*Na₂ 2H₂O 0.05%, glycerol 2.00%, α-cyclodextrin 4.00%, Kolliphor® HS15 1.00%, poloxamer 407 0.80%, Vitamin E-TPGS 0.079%, and HCl/NaOH q.s. for pH adjustment in purified water.

All the evaluated drugs, that have low aqueous solubility and Log P values ranging from 1.9 to 7.5, were formulated as aqueous eye drop lipid microsuspension. In the vehicle they all formed a lipid microsuspension, with the right appearance, a suitable particle size profile, an acceptable rheological behavior, and with no visible impact on the osmolality (Table 9). Thus, the aqueous eye drop lipid microsuspension can be used to deliver wide variety of lipophilic drugs.

TABLE 9
Physiochemical characteristics of lipid microsuspensions containing different drugs.

| | | | | | Cyclosporin | Loteprednol | | No |
|---|---|---|---|---|---|---|---|---|
| Parameter | | Everolimus | Tacrolimus | Sirolimus | A | etabonate | Dexamethasone | drug |
| Drug LogP | | 5.9 | 2.7 | 6.0 | 7.5 | 3.9 | 1.9 | - |
| Drug conc. (% w/w) | | 0.05 | 0.05 | 0.05 | 0.1 | 0.05 | 0.05 | - |
| pH | | 5.2 | 5.0 | 5.9 | 5.9 | 6.5 | 6.4 | 5.3 |
| Osmolality | | 291 | 304 | 309 | 292 | 282 | 297 | 293 |
| (mOsmol/kg) | | | | | | | | |
| Viscosity at 15 | | 19.4 | 15.7 | 16.5 | 13.6 | 16.1 | 17.7 | 8.7 |
| rpm (cP) | | | | | | | | |
| Particle | Dx(10) | 1.6 | 1.5 | 1.5 | 2.4 | 1.5 | 1.4 | 1.3 |
| size | Dx(50) | 6.6 | 5.3 | 5.7 | 5.5 | 6.2 | 4.6 | 4.3 |
| (μm) | Dx(90) | 26 | 22 | 25 | 36 | 24 | 16 | 17 |

### Example 21: The Effect of Polyols on the Physiochemical Characteristics of the Eye Drops

Eye drops were prepared as described in Example 20 but where glycerol was either omitted or replaced by another type of polyol. The composition of the aqueous lipid microsuspension vehicle was as follows: Castor oil 3.00%, EDTA*Na₂ 2H₂O 0.05%, α-cyclodextrin 4.00%, Kolliphor® HS15 1.00%, poloxamer 407 0.80%, Vitamin E-TPGS 0.079%, and HCl/NaOH q.s. for pH adjustment in purified water. The formulation contained no polyol, 2.00% glycerol, 2.00% sorbitol, 2.00% propylene glycol (PG) or 2.00% polyethylene glycol 400 (PEG 400) (all % w/w). Glycerol shows the best profile, e.g. in terms of osmolality and particle size distribution.

TABLE 10
Physiochemical characteristics of lipid microsuspensions
containing different polyols.

| Sample | | | | | | |
|---|---|---|---|---|---|---|
| Parameter | | No polyol | Glycerol | Sorbitol | PG | PEG 400 |
| pH | | 5.3 | 5.3 | 5.4 | 5.3 | 5.2 |
| Osmolality | | 27 | 293 | 169 | 334 | 80 |
| (mOsmol/kg) | | | | | | |
| Viscosity at 15 | | 12 | 9 | 12 | 18 | 7 |
| rpm (cP) | | | | | | |
| Particle | Dx(10) | 1.3 | 1.3 | 1.5 | 1.4 | 1.4 |
| size | Dx(50) | 5.6 | 4.2 | 8.5 | 4.4 | 12 |
| (μm) | Dx(90) | 17 | 17 | 31 | 17 | 71 |

| | | | | | |
|---|---|---|---|---|---|
| Particle size | Bimodal | Unimodal | Bimodal | Unimodal | Bimodal |
| distribution | | | | | |
| Delta | 21.2 | 17.1 | 19.4 | 19.4 | 25.0 |
| sedimentation | | | | | |
| (%)* | | | | | |

*Relative segregation reported as the delta percentage upon sample centrifugation at 4,500 rpm and 13,500 rpm/Lower values are best.

### Example 22: In Vivo Testing in Rabbits

The objectives of this study were to compare the concentration of everolimus in ocular tissues and to evaluate potential irritation following single administration of 0.05% everolimus eye drops in the conjunctival sac of the left eye of female New Zealand White rabbits. An aqueous 0.05% everolimus eye drop lipid microsuspension was prepared as described in Example 20. The composition the eye drops were as follows (all w/w %): 0.05% everolimus in a vehicle composed of castor oil 3.00%, EDTA*Na₂ 2H₂O 0.05%, glycerol 2.00%, α-cyclodextrin 4.00%, Kolliphor® HS15 1.00%, Poloxamer 407 0.80%, and Vitamin E-TPGS 0.079% in purified water. The pH was adjusted to 5.4 with HCl/NaOH. The osmolality of the eye drops was 291 mOsmol/Kg.

One drop (30 μl) was administered to the left eye and the levels of the drug measured 2 hours after the administration. Six female rabbits were used and the results are the mean values±standard deviation. The right eye served as a reference eye. The everolimus concentrations were measured in both eyes in the cornea, aqueous humor, vitreous humor, sclera, and plasma. Levels of everolimus in plasma and ocular tissue supernatants were quantified using an RGA-2 (Research Grade Assay-2) LC-MS/MS method, using a surrogate matrix-matched calibration curve and QC samples. Surrogate matrix suitability experiments have been performed to demonstrate the justified use of this approach. RGA-2 criteria: bracketing curves with overall, at least 75% of the calibration samples must back-calculate to within 25% (30% at LLOQ (lower limit of quantitation)) of their actual concentration and at least 66% of the QC samples must be within 25% of their actual concentration. For analysis of everolimus, an aliquot of each prepared sample was injected onto the HPLC column by an automated sample injector (SIL30-AC, Shimadzu, Japan). Chromatographic separation was performed on a Kinetix® C18 column (2.1×50 mm, 2.6 μm; Phenomenex) held at a temperature of 50 °C. Components were separated using a gradient of ultra-purified water and methanol, both containing 0.1% trifluoracetic acid, at a flow rate of 0.4 mL/min. The MS analyses were performed using an API 4500 MS/MS system (Sciex, USA). The instrument was operated in multiple-reaction-monitoring (MRM) mode. The acquisitions were performed in positive ionization mode, with optimized settings for test items. Data were acquired and processed using the Analyst™ data system (v 1.7.2, Sciex, USA).

TABLE 11
Concentration of 0.05% (w/w) everolimus in ocular tissues two
hours after topical administration of aqueous everolimus
lipid microsuspension.

| | Everolimus concentration | |
|---|---|---|
| | (pmol/g), mean ± | |
| Tissue | standard deviation | LLOQ* |
| Cornea | 91.6 ± 24.6 | 0.8 pmol/g |
| Conjunctiva | 159 ± 103 | 0.8 pmol/g |
| Sclera | 7.34 ± 1.49 | 0.8 pmol/g |
| Aqueous humor | <0.45 | 0.4 pmol/g |
| Vitreous humor | Below LLOQ | 0.4 pmol/g |
| Plasma | Below LLOQ | 0.2 nM |

*Lower limit of quantitation (LLOQ)

The everolimus levels in the reference eye (i.e., the right eye) were below the LLOQ. Treatment with the 0.05% everolimus eye drops was well tolerated and the study showed that the irritating potential of 0.05% everolimus eye drop lipid microsuspension is negligible. Quantifiable concentrations of everolimus were observed in aqueous humor, cornea, sclera, and conjunctiva, but not in vitreous humor and plasma. The highest everolimus concentrations were found in the conjunctiva, ranging from 47.6 to 308 μmol/g. The cornea contained the second highest concentrations, ranging from 67.2 to 127 μmol/g, while the other ocular tissues contained notably lower everolimus concentrations. Thus, the 0.05% everolimus eye drop lipid microsuspension targets the anterior eye tissues making it ideal for therapeutic treatment of anterior ocular conditions.

### Example 23: Process with High-Shear Silverson Homogenizer

The formulation manufacturing process as described in Example 17 can be implemented in an optimized way with a high-shear L5M Silverson homogenizer using the “square hole screen” probe (Silverson reference: 7250-HQ0005) operated at 9,000 RPM for 9 minutes.

TABLE 12
Formulation composition and physiochemical characteristics.

| Ingredient | Quantitative formula (% w/w) |
|---|---|
| Everolimus | 0.05 |
| α-Cyclodextrin | 4.00(*) |
| Castor oil | 3.0 |
| Glycerol | 2.0 |
| Kolliphor HS 15 | 1.0 |
| Poloxamer 407 | 0.8 |
| EDTA, Na₂, 2 H₂O | 0.05 |
| Purified water | Q.S.100 |
| Sedimentation rate | 52.0% |
| Particles | |
| Dx50 (μm) | 1.1 |
| Dx10 (μm) | 2.5 |
| Dx90 (μm) | 7.9 |

(*) Amount corrected for water content

The process with L5M Silverson homogenizer enables production of lipid microsuspension with improved homogeneity and smaller particles.

### Example 24: The Stabilizing Effect of EDTA

Two everolimus formulations were prepared according to the process described in Example 17 using the Ultra-Turrax® T25 homogenizer operated 12,000 RPM for about 10 minutes. One contained no EDTA (Formulation A) while the other one contained 0.05% (w/w) of EDTA (Formulation B). The formulations were stored at 40 °C for 3 months in glass containers and loss of everolimus, pH changes and the amount of total impurities monitored (Table 13). Also, the concentration on butylated hydroxytoluene (BHT) in Formulation A was significantly reduced upon storage while the BHT concentration in Formulation B was constant. Including EDTA in the formulation stabilized the pH and reduced everolimus degradation during storage.

TABLE 13
Formulation composition (% w/w) and physiochemical characteristics.

| | Formulation A | Formulation B |
|---|---|---|
| Everolimus (including 2% BHT) | 0.05% | 0.05% |
| Castor oil | 3.0% | 3.0% |
| α-Cyclodextrin (dry material)(*) | 4.0% | 4.0% |
| Glycerol | 2.0% | 2.0% |
| Kolliphor ® HS15 | 1.0% | 1.0% |
| Poloxamer 407 | 0.8% | 0.8% |
| Disodium edetate dihydrate (EDTA) | 0% | 0.05% |
| Purified water | q.s. 100 | q.s. 100 |
| Hydrochloric acid or Sodium hydroxide | q.s. pH | q.s. pH |
| After storage for 3 months at 40 °C: | 5.3 ± 0.1 | 5.3 ± 0.1 |
| pH | 4.93 | 5.27 |
| Everolimus concentration | 2.54% | 2.84% |
| Total impurities (% increase from time zero) | 872% | 831% |

(*)Amount corrected for water content.

### Example 25: Stability in Plastic Containers

Eye drops were prepared according to the process described in Example 17 using the Ultra-Turrax® T25 homogenizer operated 12,000 RPM for about 10 minutes. The composition of the aqueous lipid microsuspension eye drops was as follows: Everolimus 0.05%, castor oil 3.00%, α-cyclodextrin 4.00%, glycerol 2.0%, Kolliphor® HS15 1.00%, poloxamer 407 0.80%, disodium edetate dihydrate (EDTA) 0.05%, and HCl/NaOH q.s. for pH adjustment to 5.3 in purified water. The formulation was stored in primary plastic containers, that is ophthalmic plastic bottles with droppers and caps made of LDPE (low density polyethylene of pharmaceutical grade), 5 ml bottle size, fully filled by the formulation). Two types of sterilization methods were applied, by gamma-radiation at 25 kGy (Container A) and by treatment with ethylene oxide (Container B).

TABLE 14
Physiochemical characterization of the eye drops during storage.

| | | | | 25° C. and 60% | | 40° C. and <25% | |
|---|---|---|---|---|---|---|---|
| Storage conditions: | | 5° C. (± 3° C.) | | relative humidity | | relative humidity | |
| Container: | | A | B | A | B | A | B |
| Storage time | | | | | | | |
| pH | | | | | | | |
| | Initial | 5.4 | 5.4 | 5.4 | 5.4 | 5.4 | 5.4 |
| | 3 months | 5.4 | 5.4 | 5.3 | 5.2 | 5.1 | 4.9 |
| | 6 months | 5.3 | 5.2 | 5.2 | 5.0 | 4.8 | 4.6 |
| Tonicity (mOsmol/kg) | | | | | | | |
| | Initial | 284 | 284 | 284 | 284 | 284 | 284 |
| | 3 months | 287 | 285 | 283 | 287 | 291 | 291 |
| | 6 months | 287 | 287 | 287 | 290 | 297 | 302 |
| Particle size (Dx50 (mm)) | | | | | | | |
| | Initial | 6.6 | 6.6 | 6.6 | 6.6 | 6.6 | 6.6 |
| | 3 months | 5.5 | 5.5 | 6.6 | 6.2 | 2.7 | 2.8 |
| | 6 months | 5.8 | 5.4 | 6.2 | 6.2 | 2.6 | 2.6 |
| PSD profile (*) | | | | | | | |
| | Initial | Complies | Complies | Complies | Complies | Complies | Complies |
| | 3 months | Complies | Complies | Complies | Complies | Complies | Complies |
| | 6 months | Complies | Complies | Complies | Complies | Complies | Complies |
| Everolimus assay | | | | | | | |
| (HPLC) | | | | | | | |
| (% of Label Claim | | | | | | | |
| 0.05% w/w) | | | | | | | |
| | Initial | 100.0 | 100.0 | 100.0 | 100.0 | 100.0 | 100.0 |
| | 3 months | 97.3 | 97.3 | 95.6 | 96.6 | 94.1 | 93.9 |
| | 6 months | 97.0 | 96.0 | 94.5 | 94.3 | 88.2 | 88.1 |
| Total impurities assay | | | | | | | |
| (HPLC) (%) | | | | | | | |
| | Initial | 0.2 | 0.2 | 0.2 | 0.2 | 0.2 | 0.2 |
| | 3 months | 0.3 | 0.3 | 0.8 | 0.7 | 3.0 | 2.6 |
| | 6 months | 0.7 | 0.5 | 2.0 | 1.9 | 6.5 | 6.3 |

(*): Particle size distribution profile:
“Complies” means: “similar as initial time with Gaussian style plot”.

1. An ophthalmic composition, comprising:
at least one mTOR inhibitor as an active pharmaceutical ingredient;
a cyclodextrin and
an oil.

2. The ophthalmic composition according to claim 1, wherein the cyclodextrin is α-cyclodextrin.

3. The ophthalmic composition according to claim 1, wherein the ophthalmic composition is a microsuspension, and wherein the oil and the cyclodextrin form microparticles in an aqueous vehicle.

4. The ophthalmic composition according to claim 3, wherein the microparticles have a diameter D50 of less than 40 μm.

5. The ophthalmic composition according to claim 1, further comprising glycerol as a tonicity agent.

6. The ophthalmic composition according to claim 1, further comprising poloxamer 407 as a polymer.

7. The ophthalmic composition according to claim 1, further comprising a stabilizing agent selected from the group consisting of polyoxyethylene castor oil derivatives, and polyoxyl 15 hydroxystearate.

8. The ophthalmic composition according to claim 1 prepared as a microsuspension, comprising:
0.01 to 0.1% of everolimus;
3 to 5% of α-cyclodextrin;
2 to 4% of castor oil;
1 to 3% of glycerol;
0.5 to 2% of a stabilizing agent;
0.4 to 1.2% of a polymer;
0 to 0.07% of a complexing agent; and
0 to 0.5% of an antioxidant,
wherein percentages are weight based on a weight of the ophthalmic composition.

9. A method of preparing an ophthalmic microsuspension, comprising:
a) preparing an aqueous composition A by dissolving α-cyclodextrin into purified water;
b) preparing an oil phase composition B comprising mTOR inhibitor;
c) optionally sterilizing the oil phase composition B;
d) adding the oil phase composition B to the aqueous composition A to obtain a mixture C; and
e) homogenizing the mixture C to obtain a microsuspension.

10. The method of preparing an ophthalmic composition according to claim 9, comprising:
a) preparing the composition A by dissolving α-cyclodextrin in purified water;
b) adding at least one tonicity agent, and at least one complexing agent, to the composition A;
c) autoclaving the composition A of step b);
d) adding an aqueous solution of concentrated stabilizing agent to the composition of step c);
e) dissolving the mTOR inhibitor, e.g. everolimus, in an oil, obtain the composition B;
f) adding the composition B through a filter to the composition of step e) to obtain the mixture C;
g) homogenizing the mixture C) to obtain the microsuspension;
h) adding an aqueous solution of concentrated polymer to the microsuspension;
i) adjusting the pH of the microsuspension to a selected pH; and
j) if required, adjusting a final volume or weight of the formulation by adding water.

11. An ophthalmic microsuspension obtained by the method according to claim 9.

12. A method for treating an ocular condition by administration of the ophthalmic composition of claim 1 to a subject in need thereof.

13. A method for preventing rejection in corneal transplantation, wherein the composition of claim 1 is topically administered to an eye in an amount of 1 drop, one to 3 times per day for 2 to at least 12 months, starting from 4 weeks after the surgical operation.

14. A method for treating dry eye disease or meibomian gland dysfunctions, wherein the composition of claim 1 is topically administered to either an eye or an eyelid in an amount of 1 drop, one to 3 times per day for 2 to at least 12 months.

15. The method according to claim 14, wherein the composition comprises 0.01 to 0.1 of mTOR inhibitor by weight based on the weight of the composition.

16. The ophthalmic composition of claim 1 wherein the MTOR inhibitor is eyerolimus,
the cyclodextrin is α-cyclodextrin, and
the oil is castor oil.

17. The ophthalmic composition of claim 4 wherein the diameter D50 is less than 10 μm.

18. The method of claim 12 wherein the ocular condition is selected from the group consisting of allergic or atopic conjunctivitis), corneal transplantation, dry eye disease (DED), meibomian gland dysfunctions, pterygium, corneal endothelial disorders, corneal dystrophies, and trabeculectomy.
